(19) United States
(12) Patent Application Publication
(10) Pub. No.: US 2010/0179185 A1
(43) Pub. Date: Jul. 15, 2010

(54) Novel Aminothienopyridinone Derivatives Processes for Preparing Them and Pharmaceutical Compositions Thereof

(75) Inventors: Jeremy Martin Davis, Slough (GB); Daniel Christopher Brookings, Slough (GB); Barry John Langham, Slough (GB); Martin Clive Hutchings, Slough (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Bruselles (BE)

(21) Appl. No.: 12/515,380

(22) PCT Filed: Nov. 23, 2007
(86) PCT No.: PCT/EP2007/010189

§ 371 Date: Nov. 19, 2009

(30) Foreign Application Priority Data
Nov. 30, 2006 (GB) 0623955.2
Jan. 27, 2007 (EP) 07001807.2

(51) Int. Cl. A61K 31/4365; C07D 495/04; A61P 29/00

(52) U.S. Cl. 514/301; 546/114

(57) ABSTRACT

The present invention concerns novel 3-aminothienopyridinone derivatives of the formula I, processes for preparing them, pharmaceutical compositions containing them and their use as pharmaceuticals.

The present invention concerns novel 3-aminothienopyridinone derivatives, processes for preparing them, pharmaceutical compositions containing them and their use as pharmaceuticals.

Mitogen activating protein (MAP) kinases are a sub-class of serine/threonine kinases which play a key role in cell signalling [Adams, J. L. et al., Progress in Medicinal Chemistry, pp. 1-60, King, F. D. and Oxford, A. W. eds., vol. 38, Elsevier Science, 2001]. p38 MAP kinase occupies a central position within the cascade of signalling molecules mediating extracellular to intracellular signalling. Its influence over not only IL-1, TNF and IL-8 production but also the synthesis and/or action of other pro-inflammatory proteins (e.g. IL-6, GM-CSF, COX-2, collagenase and stromelysin) make it an attractive target for inhibition by small molecule inhibitors with the expectation that such inhibition would be a highly effective mechanism for regulating the excessive and destructive activation of the immune system. Such an expectation is supported by the potent and diverse anti-inflammatory activities described for p38 kinase inhibitors [Adams, ibid; Badger et al., J. Pharm. Exp. Ther., 1996, 279, 1453-61; Griswold et al., Pharmacol. Comm., 1996, 7, 323-29].

Patent application WO 2004/113349 discloses the process for synthesis of compounds of formula:

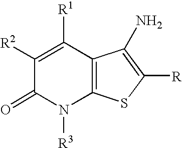

Patent application WO 2004/113348 discloses the preparation of p38 MAP kinase inhibitors of formula:

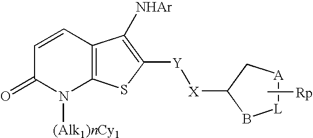

Patent application WO 2004/113347 discloses the preparation of p38 MAP kinase inhibitors of formula:

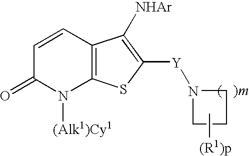

Patent application WO 2004/000846 discloses the preparation of p38 MAP kinase inhibitors of formula:

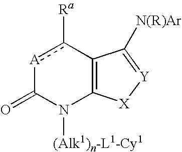

Patent application WO 2006/056412 discloses the preparation of compounds of formula:

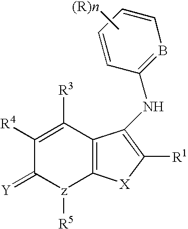

Patent application WO 2005/042540 discloses the preparation of compounds of formula

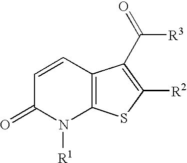

We have now discovered a group of compounds which are potent and selective inhibitors of p38 MAP kinase, especially p38α, p38β and p38β2, and splice variants thereof. The specific combination of functional groups conveys significant and unexpected advantages over compounds described in our previous patent filings. The compounds in accordance with the present invention are thus of use in medicine, for example, in the prophylaxis and treatment of immune or inflammatory disorders described herein.

In addition, the compounds according to the present invention may be used as pharmacological standards for the use in the development of new biological tests and in the search for new pharmacological agents. Thus, the compounds according to this invention may be useful as radioligands in assays for detecting compounds capable of binding to the p38 MAP kinase enzyme.

In one aspect, the invention provides a compound having formula I or pharmaceutically acceptable salts thereof or stereoisomeric forms thereof, and the geometrical isomers, enantiomers, diastereoisomers, and pharmaceutically acceptable salts thereof

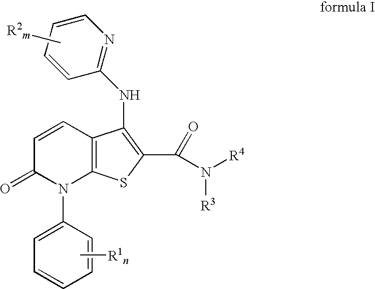

wherein

R¹ is independently C₁₋₃ alkyl, halogen or hydroxyl;
R² is independently C₁₋₃ alkyl, halogen or hydroxyl;
n is 1 to 3;
m is 1 to 3;
R³ and R⁴ form together with the nitrogen atom a 4, 5 or 6 membered non-aromatic heterocycle optionally substituted by a substituent selected from the group constituted of C₁₋₃ alkyl or hydroxyl.

The term “alkyl”, as used herein, refers to saturated, monovalent or divalent hydrocarbon radicals having linear or branched moieties and containing 1-3 carbon atoms. Alkyl groups may optionally be substituted by hydroxyl. Usually alkyl groups in the present case are methyl, hydroxymethyl.

The term “halogen”, as used herein, refers to an atom of chlorine, bromine, fluorine, iodine. Usually, halogen is fluorine.

The term “hydroxyl”, as used herein, refers to a group of formula —OH.

The term “heterocycle”, as used herein refers to a 4, 5 or 6 membered saturated ring, containing the N atom from the general structure. Heterocycles can optionally be substituted by C₁₋₃ alkyl or hydroxyl, as described above. Usually heterocycle groups in the present case are 3-(S)-hydroxypyrrolidine, 2-(S)-hydroxymethylpyrrolidine, 2-(R)-hydroxymethylpyrrolidine, 3-(R)-hydroxypyrrolidine, piperazine. Most preferred heterocycles are 2-(S)-hydroxymethylpyrrolidine, 3-(R)-hydroxypyrrolidine, 2-(R)-hydroxymethylpyrrolidine.

Generally R¹ is C₁₋₃ alkyl, halogen or hydroxyl. Usually R¹ is halogen. Preferred R¹ is fluorine.

Generally R² is C₁₋₃ alkyl, halogen or hydroxyl. Usually R² is C₁₋₃ alkyl or halogen. Preferred R² is methyl, fluorine.

Generally n is 1 to 3. Usually n is 2.

Generally m is 1 to 3. Usually m is 1 or 2.

Generally R³ and R⁴ form together with the nitrogen atom a 4, 5 or 6 membered non-aromatic heterocycle optionally substituted by a substituent selected from the group constituted of C₁₋₃ alkyl or hydroxyl. Usually R³ and R⁴ form together 3-(S)-hydroxypyrrolidine, 2-(S)-hydroxymethylpyrrolidine, 2-(R)-hydroxymethylpyrrolidine, 3-(R)-hydroxypyrrolidine or piperazine. Most preferred R³ and R⁴ together are 3-(R)-hydroxypyrrolidine, 2-(R)-hydroxymethylpyrrolidine, 2-(S)-hydroxymethylpyrrolidine.

Preferred compounds of the invention are:
7-(2,6-Difluorophenyl)-2-{[(2R)-2-(hydroxymethyl)pyrrolidin-1-yl]carbonyl}-3-[(6-methylpyridin-2-yl)amino]thieno[2,3-b]pyridin-6(7H)-one;
7-(2,6-Difluorophenyl)-2-{[(2S)-2-(hydroxymethyl)pyrrolidin-1-yl]carbonyl}-3-[(6-methylpyridin-2-yl)amino]thieno[2,3-b]pyridin-6(7H)-one;
7-(2,6-Difluorophenyl)-2-{[(3R)-3-hydroxypyrrolidin-1-yl]carbonyl}-3-[(6-methylpyridin-2-yl)amino]thieno[2,3-b]pyridin-6(7H)-one;
7-(2,6-Difluorophenyl)-2-{[(3S)-3-hydroxypyrrolidin-1-yl]carbonyl}-3-[(6-methylpyridin-2-yl)amino]thieno[2,3-b]pyridin-6(7H)-one;
7-(2,6-Difluoro-phenyl)-3-[(5-fluoro-6-methylpyridin-2-yl)amino]-2-{[(3R)-3-hydroxypyrrolidin-1-yl]carbonyl}thieno[2,3-b]pyridin-6(7H)-one;
7-(2,6-Difluorophenyl)-3-[(5-fluoro-6-methylpyridin-2-yl)amino]-2-{[(2R)-2-(hydroxymethyl)pyrrolidin-1-yl]carbonyl}thieno[2,3-b]pyridin-6(7H)-one;
7-(2,6-Difluorophenyl)-3-(6-methylpyridin-2-ylamino)-2-{[1-piperazinyl]carbonyl}thieno[2,3-b]pyridin-6(7H)-one.

Most preferred compounds of the invention are:
7-(2,6-Difluorophenyl)-2-{[(2R)-2-(hydroxymethyl)pyrrolidin-1-yl]carbonyl}-3-[(6-methylpyridin-2-yl)amino]thieno[2,3-b]pyridin-6(7H)-one;
7-(2,6-Difluorophenyl)-2-{[(3R)-3-hydroxypyrrolidin-1-yl]carbonyl}-3-[(6-methylpyridin-2-yl)amino]thieno[2,3-b]pyridin-6(7H)-one;
7-(2,6-Difluoro-phenyl)-3-[(5-fluoro-6-methylpyridin-2-yl)amino]-2-{[(3R)-3-hydroxypyrrolidin-1-yl]carbonyl}thieno[2,3-b]pyridin-6(7H)-one;
7-(2,6-Difluorophenyl)-3-[(5-fluoro-6-methylpyridin-2-yl-amino]-2-{[(2R)-2-(hydroxymethyl)pyrrolidin-1-yl]carbonyl}thieno[2,3-b]pyridin-6(7H)-one.

Compounds of formula I and some of their intermediates have at least one stereogenic centre in their structure. This stereogenic centre may be present in an R or an S configuration, said R and S notation is used in correspondence with the rules described in Pure Appl. Chem. (1976), 45, 11-3.

The “pharmaceutically acceptable salts” according to the invention include therapeutically active, non-toxic base and acid salt forms, which the compounds of formula I are able to form.

The acid addition salt form of a compound of formula I that occurs in its free form as a base can be obtained by treating the free base with an appropriate acid such as an inorganic acid, for example, a hydrohalic such as hydrochloric, hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic, formic and the like (Handbook of Pharmaceutical Salts, P. Heinrich Stahl & Camille G. Wermuth (Eds), Verlag Helvetica Chimica Acta—Zürich, 2002, 329-345).

The compounds of formula I containing acidic protons may be converted into their therapeutically active, non-toxic base addition salt forms, e.g. metal or amine salts, by treatment with appropriate organic and inorganic bases. Appropriate base salt forms include, for example, ammonium salts, alkali and earth alkaline metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like (Handbook of Pharmaceutical Salts, P. Heinrich Stahl & Camille G. Wermuth (Eds), Verlag Helvetica Chimica Acta—Zürich, 2002, 329-345). Conversely said salt forms can be converted into the free forms by treatment with an appropriate base or acid.

Compounds of formula I and their salts, can be in the form of a solvate, which is included within the scope of the present invention. Such solvates include for example hydrates, alcoholates and the like.

The invention also relates to all stereoisomeric forms such as enantiomeric and diastereoisomeric forms of the compounds of formula I or mixtures thereof (including all possible mixtures of stereoisomers).

Some of the compounds of formula I may also exist in tautomeric forms. Such forms although not explicitly indicated in the above formula are intended to be included within the scope of the present invention.

With respect to the present invention reference to a compound or compounds, is intended to encompass that compound in each of its possible isomeric forms and mixtures thereof unless the particular isomeric form is referred to specifically.

Compounds according to the present invention may exist in different polymorphic forms. Although not explicitly indicated in the above formula, such forms are intended to be included within the scope of the present invention.

The present invention concerns also processes for preparing the compounds of formula I.

When compounds of formula I present one stereogenic centre, and that non-stereoselective methods of synthesis are used, resolution of the mixture of stereoisomers can best be effected in one or several steps, involving generally sequential separation of mixtures of diastereomers into their constituting racemates, using preferably chromatographic separations on achiral or chiral phase in reversed or preferably in direct mode, followed by at least one ultimate step of resolution of each racemate into its enantiomers, using most preferably chromatographic separation on chiral phase in reversed or preferably in direct mode. Alternatively, when partly stereoselective methods of synthesis are used, the ultimate step may be a separation of diastereomers using preferably chromatographic separations on achiral or chiral phase in reversed or preferably in direct mode.

It has now been found that compounds of formula I and their pharmaceutically acceptable salts are useful to the prophylaxis or treatment of any disease or disorder in which p38 MAP kinase plays a role including conditions caused by excessive or unregulated pro-inflammatory cytokine production including for example excessive or unregulated TNF, IL-1, IL-6 and IL-8 production in a human, or other mammal.

Compounds of this invention also exhibit inhibition of expression of inducible pro-inflammatory proteins such as prostaglandin endoperoxidase synthetase-2, otherwise known as cyclooxygenase-2 (COX-2), and are therefore of use in therapy.

For example, the compounds according to the invention are useful for the treatment or prevention of inflammatory and immune disorders, cardiovascular disease, fibrotic diseases, transplantation, destructive bone disorders, neurodegenerative diseases, infectious diseases, pain and cancers. Examples of such conditions are inflammatory diseases including Crohn's disease, ulcerative colitis, chronic obstructive pulmonary disease, nephritis and hepatitis; allergies and hypersensitivity reactions including asthma, allergic rhinitis, sinusitis, conjunctivitis, food allergy, dermatitis, psoriasis, urticaria, pruritis and eczema; autoimmune diseases including rheumatoid arthritis, psoriatic arthritis, systemic lupus, erythematosis and multiple sclerosis; ischaemic heart disease and vascular diseases including atherosclerosis and arteritis; fibrotic disease including cirrhosis, renal fibrosis, adhesions and scarring; transplantation including renal, cardiac and liver; destructive bone disorders such as osteoporosis, osteoarthritis and multiple myeloma-related bone disease; neurodegenerative diseases such as Alzheimer's disease and cerebral ischemias; infectious diseases such as septic shock, sepsis and Shigellosis; viral diseases such as acute hepatitis infection and HIV infection; pain such as neuromuscular pain, headache, dental pain, arthritis pain and pain caused by cancer; cancers such as acute or chronic myelogenous leukemia, Kaposi's sarcoma, melanoma, multiple myeloma, lung, pancreatic, prostate, renal, liver, cervical, ovarian, mammary carcinoma, endometrial, bladder, seminomas, thyroid, and gastric cancer; treatment of colorectal cancer and pre-cancerous lesions, e.g. adenomatous polyposis, leading to colon cancer; treatment of oesophageal cancer including Barrett's oesophagus and it's progression to adenocarcinoma.

Thus, the present invention, in a further aspect, concerns the use of a compound of formula I or a pharmaceutically acceptable salt thereof for the manufacture of a medicament for the treatment of disorders such as mentioned above.

The present invention concerns the use of a compound of formula I or a pharmaceutically acceptable salt thereof, for the manufacture of a medicament for the treatment of inflammatory diseases including Crohn's disease, ulcerative colitis, chronic obstructive pulmonary disease, nephritis and hepatitis; allergies and hypersensitivity reactions including asthma, allergic rhinitis, sinusitis, conjunctivitis, food allergy, dermatitis, psoriasis, urticaria, pruritis and eczema; autoimmune diseases including rheumatoid arthritis, psoriatic arthritis, systemic lupus, erythematosis and multiple sclerosis; ischaemic heart disease and vascular diseases including atherosclerosis and arteritis; fibrotic disease including cirrhosis, renal fibrosis, adhesions and scarring; transplantation including renal, cardiac and liver; destructive bone disorders such as osteoporosis, osteoarthritis and multiple myeloma-related bone disease; neurodegenerative diseases such as Alzheimer's disease and cerebral ischemias; infectious diseases such as septic shock, sepsis and Shigellosis; viral diseases such as acute hepatitis infection and HIV infection; pain such as neuromuscular pain, headache, dental pain, arthritis pain and pain caused by cancer; cancers such as acute or chronic myelogenous leukemia, Kaposi's sarcoma, melanoma, multiple myeloma, lung, pancreatic, prostate, renal, liver, cervical, ovarian, mammary carcinoma, endometrial, bladder, seminomas, thyroid, and gastric cancer; treatment of colorectal cancer and pre-cancerous lesions, e.g. adenomatous polyposis, leading to colon cancer; treatment of oesophageal cancer including Barrett's oesophagus and it's progression to adenocarcinoma. Preferred examples of such conditions are inflammatory diseases including Crohn's disease, ulcerative colitis, chronic obstructive pulmonary disease; psoriasis; autoimmune diseases including rheumatoid arthritis, systemic lupus, erythematosis and multiple sclerosis; destructive bone disorders such as osteoporosis and osteoarthritis; viral diseases such as HIV infection; pain such as neuromuscular pain, dental pain and arthritis pain; cancers such as multiple myeloma, pancreatic, prostate and gastric cancer; treatment of colorectal cancer and pre-cancerous lesions.

The compounds of the invention are useful for the treatment by administering to the patient an effective amount of one or more of the above-identified compounds or a pharmaceutically acceptable derivative or salt thereof in a pharmaceutically acceptable carrier or diluent to reduce formation of oxygen radicals. The active materials can be administered by any appropriate route, for example, orally, parenterally, intravenously, intradermally, subcutaneously, intramuscularly or topically, in liquid, cream, gel or solid form, via a buccal or nasal spray, or aerosol, a patch or suppositories. The invention further concerns the use of the compounds of formula I for the manufacture of a medicament for therapeutic application. In particular, the invention concerns the use of the compounds of formula I for the manufacture of a medicament useful for treating conditions in which p38 MAP kinase plays a role including conditions caused by excessive or unregulated pro-inflammatory cytokine production including for example excessive or unregulated TNF, IL-1, IL-6 and IL-8 production.

The invention concerns the use of the compound of formula I for the manufacture of a medicament useful for treating inflammatory diseases including Crohn's disease, ulcerative colitis, chronic obstructive pulmonary disease, nephritis and hepatitis; allergies and hypersensitivity reactions including asthma, allergic rhinitis, sinusitis, conjunctivitis, food allergy, dermatitis, psoriasis, urticaria, pruritis and eczema; autoimmune diseases including rheumatoid arthritis, psoriatic arthritis, systemic lupus, erythematosis and multiple sclerosis; ischaemic heart disease and vascular diseases including atherosclerosis and arteritis; fibrotic disease including cirrhosis, renal fibrosis, adhesions and scarring; transplantation including renal, cardiac and liver; destructive bone disorders such as osteoporosis, osteoarthritis and multiple myeloma-related bone disease; neurodegenerative diseases such as Alzheimer's disease and cerebral ischemias; infectious diseases such as septic shock, sepsis and Shigellosis; viral diseases such as acute hepatitis infection and HIV infection; pain such as neuromuscular pain, headache, dental pain, arthritis pain and pain caused by cancer; cancers such as acute or chronic myelogenous leukemia, Kaposi's sarcoma, melanoma, multiple myeloma, lung, pancreatic, prostate, renal, liver, cervical, ovarian, mammary carcinoma, endometrial, bladder, seminomas, thyroid, and gastric cancer; treatment of colorectal cancer and pre-cancerous lesions, e.g. adenomatous polyposis, leading to colon cancer; treatment of oesophageal cancer including Barrett's oesophagus and it's progression to adenocarcinoma. Preferred examples of such conditions are inflammatory diseases including Crohn's disease, ulcerative colitis, chronic obstructive pulmonary disease; psoriasis; autoimmune diseases including rheumatoid arthritis, systemic lupus, erythematosis and multiple sclerosis; destructive bone disorders such as osteoporosis and osteoarthritis; viral diseases such as HIV infection; pain such as neuromuscular pain, dental pain and arthritis pain; cancers such as multiple myeloma, pancreatic, prostate and gastric cancer; treatment of colorectal cancer and pre-cancerous lesions.

The invention further concerns the compounds of formula I for use as medicaments. The invention concerns the compounds of formula I for use as a medicament for treating inflammatory diseases including Crohn's disease, ulcerative colitis, chronic obstructive pulmonary disease, nephritis and hepatitis; allergies and hypersensitivity reactions including asthma, allergic rhinitis, sinusitis, conjunctivitis, food allergy, dermatitis, psoriasis, urticaria, pruritis and eczema; autoimmune diseases including rheumatoid arthritis, psoriatic arthritis, systemic lupus, erythematosis and multiple sclerosis; ischaemic heart disease and vascular diseases including atherosclerosis and arteritis; fibrotic disease including cirrhosis, renal fibrosis, adhesions and scarring; transplantation including renal, cardiac and liver; destructive bone disorders such as osteoporosis, osteoarthritis and multiple myeloma-related bone disease; neurodegenerative diseases such as Alzheimer's disease and cerebral ischemias; infectious diseases such as septic shock, sepsis and Shigellosis; viral diseases such as acute hepatitis infection and HIV infection; pain such as neuromuscular pain, headache, dental pain, arthritis pain and pain caused by cancer; cancers such as acute or chronic myelogenous leukemia, Kaposi's sarcoma, melanoma, multiple myeloma, lung, pancreatic, prostate, renal, liver, cervical, ovarian, mammary carcinoma, endometrial, bladder, seminomas, thyroid, and gastric cancer; treatment of colorectal cancer and pre-cancerous lesions, e.g. adenomatous polyposis, leading to colon cancer; treatment of oesophageal cancer including Barrett's oesophagus and it's progression to adenocarcinoma. Preferred examples of such conditions are inflammatory diseases including Crohn's disease, ulcerative colitis, chronic obstructive pulmonary disease; psoriasis; autoimmune diseases including rheumatoid arthritis, systemic lupus, erythematosis and multiple sclerosis; destructive bone disorders such as osteoporosis and osteoarthritis; viral diseases such as HIV infection; pain such as neuromuscular pain, dental pain and arthritis pain; cancers such as multiple myeloma, pancreatic, prostate and gastric cancer; treatment of colorectal cancer and pre-cancerous lesions.

The activity and properties of the active compounds, oral availability and stability in vitro or in vivo can vary significantly among the optical isomers of the disclosed compounds.

In a preferred embodiment, the active compound is administered in an enantiomerically enriched form, i.e., substantially in the form of one isomer

The present invention also concerns a method for treating p38 MAP kinase dependent inflammatory or medical conditions, inhibition of expression of inducible pro-inflammatory proteins such as prostaglandin endoperoxidase synthetase-2, otherwise known as cyclooxygenase-2 (COX-2), as mentioned above in a mammal in need of such treatment, comprising administering a therapeutic dose of at least one compound of formula I or a pharmaceutically acceptable salt thereof to a patient.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 0.01 to 2000 mg, preferably 0.05 to 500 mg of active ingredient per unit dosage form.

The term “treatment” as used herein includes curative treatment and prophylactic treatment. By “curative” is meant efficacy in treating a current symptomatic episode of a disorder or condition. By “prophylactic” is meant prevention of the occurrence or recurrence of a disorder or condition.

The term “substantially” as used herein refers to a composition of or higher than 95% of one isomer.

The compounds of the invention and their pharmaceutically acceptable salts are useful to the prophylaxis or treatment of any disease or disorder in which p38 MAP kinase plays a role including conditions caused by excessive or unregulated pro-inflammatory cytokine production including for example excessive or unregulated TNF, IL-1, IL-6 and IL-8 production in a human, or other mammal, inhibition of expression of inducible pro-inflammatory proteins such as prostaglandin endoperoxidase synthetase-2 and the invention extends to such a use and to the use of the compounds for the manufacture of a medicament for treating such diseases or disorders.

Diseases or disorders of this type include inflammatory and immune disorders, cardiovascular disease, fibrotic diseases, transplantation, destructive bone disorders, neurodegenerative diseases, infectious diseases, pain and cancers. Examples of such conditions are inflammatory diseases including Crohn's disease, ulcerative colitis, chronic obstructive pulmonary disease, nephritis and hepatitis; allergies and hypersensitivity reactions including asthma, allergic rhinitis, sinusitis, conjunctivitis, food allergy, dermatitis, psoriasis, urticaria, pruritis and eczema; autoimmune diseases including rheumatoid arthritis, psoriatic arthritis, systemic lupus, erythematosis and multiple sclerosis; ischaemic heart disease and vascular diseases including atherosclerosis and arteritis; fibrotic disease including cirrhosis, renal fibrosis, adhesions and scarring; transplantation including renal, cardiac and liver; destructive bone disorders such as osteoporosis, osteoarthritis and multiple myeloma-related bone disease; neurodegenerative diseases such as Alzheimer's disease and cerebral ischemias; infectious diseases such as septic shock, sepsis and Shigellosis; viral diseases such as acute hepatitis infection and HIV infection; pain such as neuromuscular pain, headache, dental pain, arthritis pain and pain caused by cancer; cancers such as acute or chronic myelogenous leukemia, Kaposi's sarcoma, melanoma, multiple myeloma, lung, pancreatic, prostate, renal, liver, cervical, ovarian, mammary carcinoma, endometrial, bladder, seminomas, thyroid, and gastric cancer; treatment of colorectal cancer and pre-cancerous lesions, e.g. adenomatous polyposis, leading to colon cancer; treatment of oesophageal cancer including Barrett's oesophagus and it's progression to adenocarcinoma.

Results obtained with compounds of formula I are indicative of a strong pharmacological effect.

For treating diseases, compounds of formula I or their pharmaceutically acceptable salts, may be employed at an effective daily dosage and administered in the form of a pharmaceutical composition.

Therefore, another embodiment of the present invention concerns a pharmaceutical composition comprising an effective amount of a compound of formula I or a pharmaceutically acceptable salt thereof in combination with a pharmaceutically acceptable diluent or carrier.

To prepare a pharmaceutical composition according to the invention, one or more of the compounds of formula I or a pharmaceutically acceptable salt thereof, is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral, topical or inhaled. Pharmaceutical compositions comprising compounds according to the invention can, for example, be administered orally or parenterally, i.e., intravenously, intradermally, intramuscularly, subcutaneously or intrathecally.

Pharmaceutical compositions suitable for oral administration can be solids, powders or liquids and can, for example, be in the form of tablets, pills, dragees, gelatine capsules, solutions, patches, suppositories, syrups, sprays, and the like. To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers. In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula I in compositions for oral administration is at least 0.5% by weight and can be up to 80% by weight with respect to the total weight of the composition.

For the preferred oral compositions, the daily dosage is in the range 0.01 to 2000 milligrams (mg) of compounds of formula I.

In compositions for parenteral administration, the quantity of compound of formula I present is at least 0.5% by weight and can be up to 33% by weight with respect to the total weight of the composition. For the preferred parenteral compositions, the dosage unit is in the range 0.01 mg to 2000 mg of compounds of formula I.

The daily dose can fall within a wide range of dosage units of compound of formula I and is generally in the range 0.01 to 2000 mg. However, it should be understood that the specific doses could be adapted to particular cases depending on the individual requirements, at the physician's discretion.

The compounds of the invention may be co-administered with other therapeutic agents. “Co-administration” in this context means the dosing either of components, which are formulated together as a single dosage form; or the administration of separately formulated agents at substantially the same time, or sequentially. In this context suitable examples of therapeutic agents may include, but are not limited to, anti-allergics e.g. histamine H1 antagonists such as cetirizine, histamine H2 antagonists, histamine H3 antagonists, anti-rheumatics e.g. gold therapies; immunomodulators e.g. methotrexate, cyclosporin, lefluonomide, IMPDH or dihydroorotate dehydrogenase inhibitors such as mycophenolate mofetil; corticosteroids e.g. prednisolone; non-steroidal anti-inflammatories (NSAIDS); leukotriene antagonists; PDE4 inhibitors such as 3-cyclo-propylmethoxy-4-difluoromethoxy-N-[3,5-di-chloropyrid-4-yl]-benzamide; muscarinic M3 antagonists; β2 agonists; theophylline; sodium cromoglycate; biologicals including anti-cytokine antibodies e.g. anti-TNF antibodies such as certolizumab pegol or adalimumab, anti-IL-6 antibodies, anti-IL17 antibodies, anti-co-stimulatory antibodies, anti-adhesion antibodies; adhesion molecule inhibitors; other inhibitors of cytokine synthesis; other kinase inhibitors e.g. sorafenib, imatinib mesylate, sunitinib malate, erlotinib, bevacizumab, PI3 kinase inhibitors and cancer chemotherapeutics.

The present invention concerns also processes for preparing the compounds of formula I.

The compounds of formula I according to the invention can be prepared analogously to conventional methods as understood by the person skilled in the art of synthetic organic chemistry.

The compounds of formula I according to the invention can also be prepared analogously to methods disclosed in WO2004/113347 and WO2004/113348.

The following processes description sets forth certain synthesis routes in an illustrative manner. Other alternative and/or analogous methods will be readily apparent to those skilled in this art.

The compounds covered by formula I can be prepared by the following general processes described below. In scheme (1) thioamides of formula (2) are prepared by reacting an isothiocyanate of formula (1) with acetonitrile in the presence of a base, e.g. sodium hexamethyldisilazane (NaHMDS), in a suitable solvent, e.g. tetrahydrofuran, optionally at a low temperature, e.g. around −78° C. up to ambient temperature.

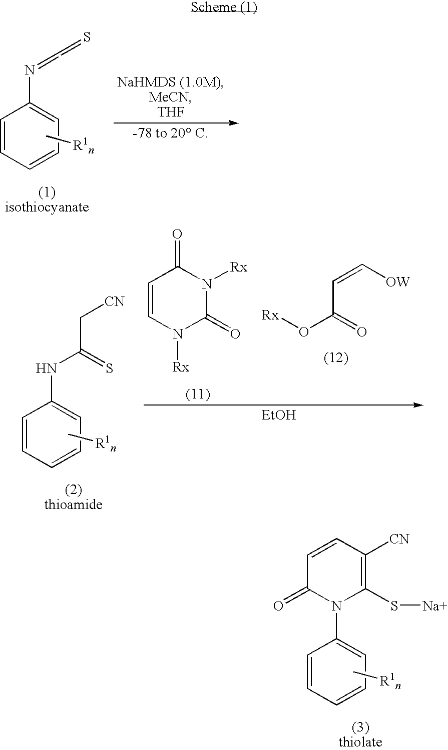

Thioamides of formula (2) can also be prepared by methods known to those skilled in the art (see, for example, Adhikari et al., Aust. J. Chem., 1999, 52, 63-67). Where isothiocyanates of formula (1) are not commercially available they may be prepared using standard methodology e.g. reaction of thiophosgene with a primary amine of formula R¹NH₂ (see review article by Drobnica et al. pp. 1003-1221, in Patai, “The Chemistry of Cyanates and Their Thio Derivatives,” pt. 2, Wiley, New York, 1977). It may be advantageous not to isolate the thioamide of formula (2) and the next reaction may be carried out directly.

Compounds of formula (3) may be prepared by reaction of thioamides of formula (2) with a compound of formula (11) or a compound of formula (12), where Rx is an optionally substituted alkyl group and W is a hydrogen atom, metal ion or amine salt. Preferably the reaction is performed with a compound of formula (11) where Rx=methyl, i.e. 1,3-dimethyl uracil. The reaction is performed in the presence of a base. Appropriate bases may include, but are not limited to, lithium bases such as n-butyl- or tert-butyllithium or lithium diisopropylamide (LDA), silazanes, e.g. lithium hexamethyldisilazane (LiHMDS) or sodium hexamethyldisilazane (NaHMDS), carbonates, e.g. potassium carbonate, alkoxides, e.g. sodium ethoxide, sodium methoxide or potassium tert-butoxide, hydroxides, e.g. sodium hydroxide (NaOH), hydrides, e.g. sodium hydride, and organic amines, e.g. triethylamine or diisopropylethylamine or a cyclic amine such as N-methylmorpholine or pyridine. The reaction may be performed in an organic solvent such as an amide, e.g. a substituted amide such as N,N-dimethylformamide, an ether, e.g. a cyclic ether such as tetrahydrofuran or 1,4-dioxane, an alcohol, e.g. methanol, ethanol or propanol, or acetonitrile, at a temperature from ambient to the reflux temperature. In one particular aspect of the process the reaction is achieved using an alkoxide base, especially sodium ethoxide or sodium methoxide, in an alcoholic solvent, especially ethanol, at reflux temperature.

Intermediates of formula (11), where not commercially available, may be prepared using standard methodology (see, for example, Mir Hedayatullah, J. Heterocyclic Chem., 1981, 18, 339). Similarly, intermediates of formula (12), where not commercially available, may be prepared using standard methodology. For example, they may be prepared in situ by reaction of an acetate, e.g. ethyl acetate, with a base such as sodium methoxide followed by addition of a formate, e.g. methyl formate.

The compounds of the invention are prepared using one of two routes from thiolate intermediate (3). In the first route (Scheme 2) thiolate (3) is reacted with an alkyl haloacetate, e.g. ethyl bromoacetate, in a suitable solvent and in the presence of a base to give 3-aminothienopyridones of formula (4).

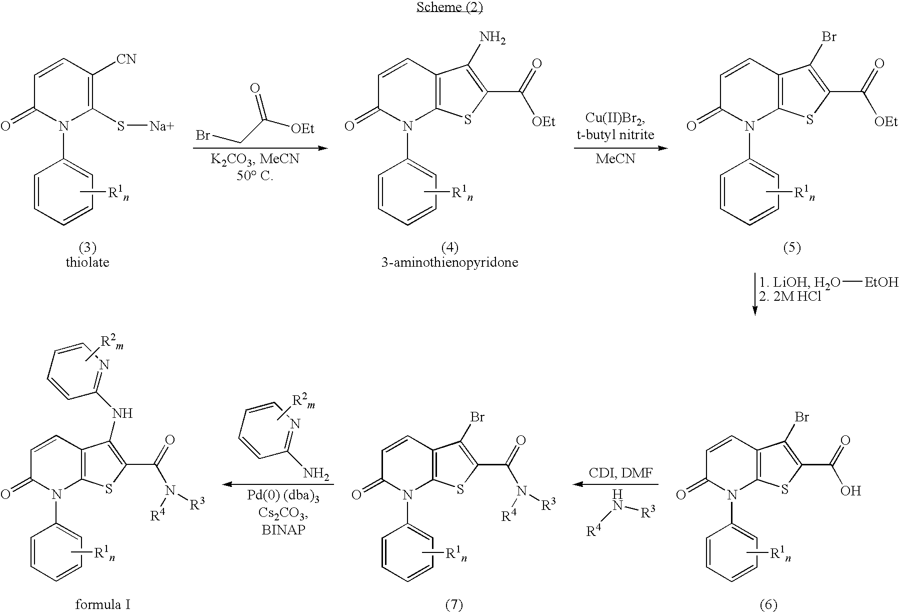

Suitable solvents include, but are not limited to, amides, e.g. a substituted amide such as N,N-dimethylformamide, alcohols, e.g. ethanol, methanol or isopropyl alcohol, ethers, e.g. a cyclic ether such as tetrahydrofuran or 1,4-dioxane and acetonitrile. The reaction may be performed at a temperature from ambient up to the reflux temperature. Appropriate bases include carbonates, e.g. caesium or potassium carbonate, alkoxides, e.g. potassium tert-butoxide, hydrides, e.g. sodium hydride, or organic amines, e.g. triethylamine or diisopropylethylamine or cyclic amines such as N-methylmorpholine or pyridine.

Aminothienopyridones of formula (4) can be converted to bromides of formula (5) by standard methods such as for example by the Sandmeyer reaction. Thus, for example, a bromide of formula (5) may be prepared by treatment of an aminothienopyridone of formula (4) with an alkyl nitrite, for example tert-butyl nitrite, and a copper salt, for example copper(II) bromide, in the presence of a solvent, for example a nitrile such as acetonitrile, at a temperature from about 0° to around 65° C. Compounds of formula (5) are hydrolysed to the corresponding carboxylic acids of formula (6) using a base such as an alkali metal hydroxide, e.g. lithium hydroxide in an alcoholic solvent such as EtOH in the presence of water. The carboxylic acid or carboxylate salt can then be converted to the desired amides of formula (7) by standard amide coupling procedures known to those skilled in art. For example, an amine of formula R⁴R³NH can be reacted with carboxylic acids of formula (6) using 1,1′-carbonyldiimidazole at room temperature in a solvent such as N,N-dimethylformamide. Note that where there is free amine or hydroxyl functionality present on the R⁴R³NH amine, then these may require protection for the amide coupling and the next step to proceed in good yield. For more details concerning protection and deprotection methods, see “Protective Groups in Organic Chemistry”, Chapter 2, J. F. W. Omie, Plenum Press, London and New York, 1973 and “Protective Groups in Organic Synthesis”, Chapter 7, Th. W. Greene, John Wiley & Sons, 1999.

The next step is the reaction of the 3-bromothienopyridones of formula (7) with a 2-pyridinamine in the presence of a palladium catalyst. The reaction may be conveniently carried out in a solvent such as toluene or an ether such as ethylene glycol dimethyl ether at an elevated temperature, e.g. the reflux temperature, using a catalyst such as tris(dibenzylideneacetone)dipalladium(0), a phosphine ligand such as 2,2′-bis(diphenylphosphino)-1,1′-binaphthyl or Xantphos, and a base such as caesium carbonate or K₃PO₄. Where desired, alternative reaction conditions may be used, for example as described in the literature [Luker et al., Tetrahedron Lett., 2001, 41, 7731; Buchwald, S. L., J. Org. Chem., 2000, 65, 1144; Hartwig, J. F., Angew. Chem. Int. Ed. Engl., 1998, 37, 2046]. After deprotection of the R⁴R³NH, if necessary, the target molecule of formula I is obtained.

Scheme (3) describes the second route used to prepare target molecules of formula I. The route is exemplified with a substituted heterocycle, but can obviously be applied to any analogue.

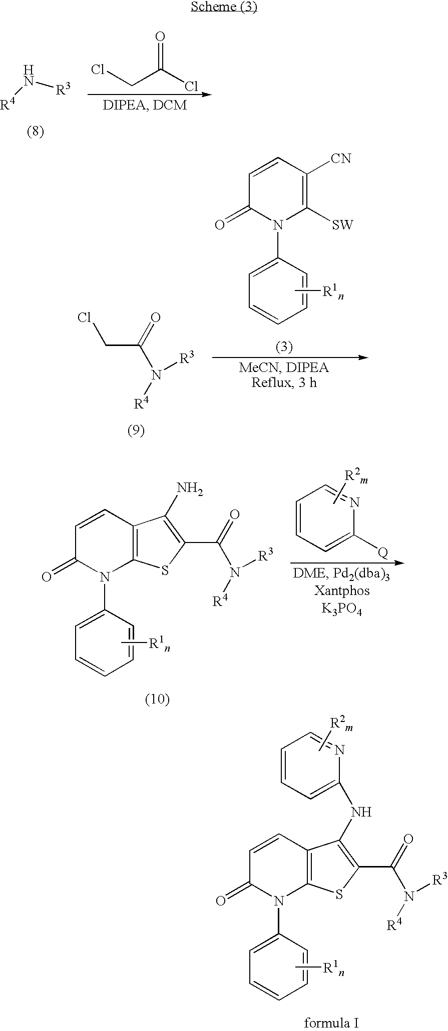

Therefore the amine of formula (8) is reacted with a halo acetylhalide, for example chloro acetylchloride, in a chlorinated solvent such as dichloromethane in the presence of a base e.g. a tertiary amine base such as diisopropylethylamine (DIPEA) at ambient temperature to give the chloromethyl amide derivative (9). It is understood that it may sometimes be necessary to protect the hydroxyl functionality, when present on (8), for this reaction and subsequent reactions to work optimally.

Reaction of compounds of formula (9) with thiolate (3) in a solvent such as acetonitrile in the presence of a base e.g. a tertiary amine base such as diisopropylethylamine DIPEA at a temperature up to the reflux temperature gives the 3-aminothienopyridones of formula (10).

Target compounds of formula I can be prepared by reaction of compounds of formula (10) with a substituted pyridine, wherein Q is a leaving group such as triflate or a halide e.g. bromine, in the presence of a transition metal catalyst e.g. a palladium catalyst. The reaction may be conveniently carried out in a solvent such as toluene or ethylene glycol dimethyl ether (DME) at an elevated temperature, e.g. the reflux temperature, using a catalyst such as tris(dibenzylideneacetone)dipalladium (0), a phosphine ligand such as 2,2′-bis(diphenylphosphino)-1,1′-binaphthyl, 4,5-bis(diphenylphosphino)-9,9-dimethylxanthene (Xantphos) or tri-tertbutylphosphine, and a base such as caesium carbonate or tripotassium phosphate. Where desired alternative reaction conditions from the literature may be used as previously described above. In an alternative procedure a copper catalyst, e.g. copper(I) iodide may be employed. The reaction may be done in the presence of a base e.g. tripotassium phosphate, optionally in a suitable solvent such as an alcohol, e.g. isopropanol, or an ether, e.g. 1,4-dioxan. A chelating ligand such as ethylene glycol or N,N-dimethylethanolamine may also be used. In reactions of this type Q is typically a halogen atom, especially an iodine atom. After deprotection of the HNR3R4 substituent, if necessary, the target compounds of formula I are obtained.

The present invention also relates to synthetic intermediates geometrical isomers, enantiomers, diastereoisomers, pharmaceutically acceptable salts and all possible mixtures thereof.

Specific synthetic intermediates are selected from the group consisting of:
Ethyl 3-amino-7-(2,6-difluorophenyl)-6-oxo-6,7-dihydrothieno[2,3-b]-pyridine-2-carboxylate;
Ethyl 3-bromo-7-(2,6-difluorophenyl)-6-oxo-6,7-dihydrothieno[2,3-b]-pyridine-2-carboxylate;
3-Bromo-7-(2,6-difluorophenyl)-6-oxo-6,7-dihydrothieno[2,3-b]pyridine-2-carboxylic acid;
3-Bromo-7-(2,6-difluorophenyl)-2-{[(2R)-2-(hydroxymethyl)pyrrolidin-1-yl]carbonyl}thieno[2,3-b]pyridin-6(7H)-one;
3-Bromo-7-(2,6-difluorophenyl)-2-({(2R)-2-[(tetrahydro-2H-pyran-2-yloxy)methyl]pyrrolidin-1-yl}carbonypthieno[2,3-b]pyridin-6(7H)-one;
3-Bromo-7-(2,6-difluorophenyl)-2-{[(2S)-2-(hydroxymethyl)pyrrolidin-1-yl]carbonyl}-thieno[2,3-b]pyridin-6(7H)-one;
3-Bromo-7-(2,6-difluorophenyl)-2-({(2S)-2-[(tetrahydro-2H-pyran-2-yloxy)methyl]pyrrolidin-1-yl}carbonyl)thieno[2,3-b]pyridin-6(7H)-one;
3-Bromo-7-(2,6-difluorophenyl)-2-{[(3R)-3-hydroxypyrrolidin-1-yl]carbonyl}thieno[2,3-b]pyridin-6(7H)-one;
3-Bromo-7-(2,6-difluorophenyl)-2-{[(3R)-3-(tetrahydro-2H-pyran-2-yloxy)pyrrolidin-1-yl]carbonyl}thieno[2,3-b]pyridin-6(7H)-one;
3-Bromo-7-(2,6-difluorophenyl)-2-{[(3S)-3-hydroxypyrrolidin-1-yl]carbonyl}-thieno[2,3-b]pyridin-6(7H)-one;
3-Bromo-7-(2,6-difluorophenyl)-2-{[(3S)-3-(tetrahydro-2H-pyran-2-yloxy)pyrrolidin-1-yl]carbonyl}thieno[2,3-b]pyridin-6(7H)-one;
3-Amino-7-(2,6-difluoro-phenyl)-2-{[(3R)-3-(tetrahydro-2H-pyran-2-yloxy)pyrrolidin-1-yl]carbonyl}thieno[2,3-b]pyridin-6(7H)-one;
7-(2,6-Difluoro-phenyl)-3-[(5-fluoro-6-methyl-pyridin-2-yl)-amino]-2-{[(3R)-3-(tetrahydro-2H-pyran-2-yloxy)pyrrolidin-1-yl]carbonyl}thieno[2,3-b]pyridin-6(7H)-one;
3-Amino-7-(2,6-difluorophenyl)-2-{[(2R)-2-(hydroxymethyl)pyrrolidin-1-yl]carbonyl}thieno[2,3-b]pyridin-6(7H)-one;
3-Amino-7-(2,6-difluorophenyl)-2-({(2R)-2-[(tetrahydro-2H-pyran-2-yloxy)methyl]pyrrolidin-1-yl}carbonyl)thieno[2,3-b]pyridin-6(7H)-one;
7-(2,6-Difluorophenyl)-3-[(5-fluoro-6-methylpyridin-2-yl)amino]-2-({(2R)-2-[(tetrahydro-2H-pyran-2-yloxy)methyl]pyrrolidin-1-yl}carbonyl)thieno[2,3-b]pyridin-6(7H)-one;
7-(2,6-Difluorophenyl)-2-{[4-(tert-butoxycarbonyl)piperazin-1-yl]carbonyl}-3-(6-methylpyridin-2-ylamino)thieno[2,3-b]pyridin-6(7H)-one;
3-Bromo-7-(2,6-difluorophenyl)-2-{[4-(tert-butoxycarbonyl)piperazin-1-yl]carbonyl}-thieno[2,3-b]pyridin-6(7H)-one;
7-(2,6-difluorophenyl)-3-{[6-(hydroxymethyl)pyridin-2-yl]amino}-2-{[(3R)-3-(tetrahydro-2H-pyran-2-yloxy)pyrrolidin-1-yl]carbonyl}thieno[2,3-b]pyridin-6(7H)-one.

The following examples are provided for illustrative purposes only and are not intended, nor should they be construed, as limiting the invention in any manner. Those skilled in the art will appreciate that routine variations and modifications of the following examples can be made without exceeding the spirit or scope of the invention.

Unless specified otherwise in the examples, characterization of the compounds is performed according to (LCMS) liquid chromatography mass spectra, preparative liquid chromatography LC, NMR, and silica gel chromatography methods. All temperatures are in ° C. The following abbreviations are used:

Sat.—saturated EtOAc—ethyl acetate
MeOH—methanol EtOH—ethanol
DCM—dichloromethane h—hour
DIPEA—diisopropylethylamine min—minute
DMSO—dimethylsulphoxide r.t.—room temperature
THF—tetrahydrofuran Et₂O—diethyl ether
DMF—N,N-dimethylformamide TBME—tert-butyl methyl ether
CDI—1,1′-carbonyldiimidazole MeCN—acetonitrile
DME—ethylene glycol dimethyl ether c.—concentrated
Pd₂(dba)₃—tris-(dibenzylideneacetone)dipalladium(0)
BINAP—2,2′-bis(diphenylphosphino)-1-1′-binaphthyl
Xantphos—4,5-bis(diphenylphosphino)-9,9-dimethylxanthene

All NMRs are obtained either at 300 MHz or 400 MHz.

Compounds are named with the aid of ACD Labs Name (v. 9.0) supplied by Advanced Chemical Development, Toronto, Canada.

LCMS retention times (RT) quoted are generated on a Hewlett Packard 1100 LC/MS using the following method: Phenomenex Luna 3μ C18(2) 50×4.6 mm column; mobile phase A=0.1% formic acid in water; mobile phase B=0.1% formic acid in MeCN; flow rate of 0.9 mLmin⁻¹, column temperature 40° C. Gradient:—

| Time (min) | % A | % B |
|---|---|---|
| 0.00 | 95.0 | 5.0 |
| 2.00 | 5.0 | 95.0 |
| 3.00 | 5.0 | 95.0 |
| 5.00 | end | |

Where stated alternative LCMS conditions (Conditions B) are used:

LCMS retention times (RT) quoted are generated on a Hewlett Packard 1100/ThermoFinnigan LCQ Duo LC/MS system using Electrospray ionisation and the following LC method: Phenomenex Luna C18(2) 5μ 100 mm×4.6 mm column; mobile phase A=0.08% formic acid in water; mobile phase B=0.08% formic acid in MeCN; flow rate of 3.0 mLmin⁻¹, column temperature 35° C. Gradient:—

| Time (min) | % A | % B |
|---|---|---|
| 0.00 | 95.0 | 5.0 |
| 4.40 | 5.0 | 95.0 |
| 5.30 | 5.0 | 95.0 |
| 5.32 | 95.0 | 5.0 |
| 6.50 | 95.0 | 5.0 |

Gas chromatographs are run on a Perkin Elmer Autosystem instrument, using an SGE 25QC2 BP5 1.0 column. Initial temperature, 70° C., heat at 15° C./min to 250° C., hold 10 min. Injector temperature 150° C., detector temperature 250° C.

### EXAMPLE 1

### Ethyl 3-amino-7-(2,6-difluorophenyl)-6-oxo-6,7-dihydrothieno[2,3-b]-pyridine-2-carboxylate (Intermediate 1)

Sodium 3-cyano-1-(2,6-difluorophenyl)-6-oxo-1,6-dihydropyridine-2-thiolate (RN 851749-68-3, 25.74 g, 90 mmol) is suspended in dry acetonitrile (200 mL). Potassium carbonate (13.5 g, 98.0 mmol) and ethyl bromoacetate (10.9 mL, 98.1 mmol) are added and the mixture heated to 50° C. for 1½ hours. The mixture is cooled to room temperature, diluted with water (600 mL) and cooled in an ice bath. The resultant solid is filtered off under vacuum, washed with water (2×100 mL) and dried in a vacuum oven at 55° C. for 18 hours to give the title compound as a white solid (27.8 g, 89%). δH (DMSO-d6) 8.27 (1H, d, J 9.7 Hz), 7.83-7.73 (1H, m), 7.51-7.46 (2H, m), 7.23 (2H, brs), 6.59 (1H, d, J 9.7 Hz), 4.15 (2H, q, J 7.1 Hz), 1.19 (3H, t, J 7.1 Hz). LCMS (ES+) RT 3.34 minutes, 351 (M+H)⁺.

### EXAMPLE 2

### Ethyl 3-bromo-7-(2,6-difluorophenyl)-6-oxo-6,7-dihydrothieno[2,3-b]-pyridine-2-carboxylate (Intermediate 2)

Intermediate 1 (13.6 g, 38.9 mmol) and copper(II) bromide (10.4 g, 46.6 mmol) are dissolved in dry acetonitrile (150 mL) and cooled to 0° C. tert-Butylnitrite (purity 90%, 7.70 ml, 58.3 mmol) is added with stirring and the reaction is left at room temperature for 18 hours. 2M HCl (75 mL) and water (150 mL) are added and the resultant solid collected by filtration and washed with water (100 mL) and dried under suction. The crude material is purified by chromatography (silica, DCM) to give the title compound as a white solid (10.9 g, 67%). δH (DMSO-d6) 8.48 (1H, s), 7.90-7.80 (1H, m), 7.60-7.52 (2H, m), 4.28 (2H, q, J 7.1 Hz), 1.25 (3H, t, J 7.1 Hz). LCMS (ES+) RT 4.27 minutes, 414/416 (M+H)⁺

### EXAMPLE 3

### 3-Bromo-7-(2,6-difluorophenyl)-6-oxo-6,7-dihydrothieno[2,3-b]pyridine-2-carboxylic acid (Intermediate 3)

Intermediate 2 (9.50 g, 23.0 mmol) is dissolved in ethanol (200 mL). Water (100 mL) and LiOH.H₂O (1.06 g, 25.2 mmol) is added. The mixture is stirred at room temperature for 18 hours. Ethanol is removed under vacuum on a rotary evaporator and the concentrated aqueous mixture is treated with 2M HCl (20 mL) and diluted with water (100 mL). The resultant solid is collected by filtration and dried in a vacuum oven at 50° C. for 72 hours to give the title product as a white solid (8.73 g, 98%). δH (DMSO-d6) 13.87 (1H, brs), 8.00 (1H, d, J 9.7 Hz), 7.86-7.78 (1H, m), 7.56-7.52 (2H, m), 6.76 (1H, d, J 9.7 Hz). LCMS (ES+) RT 3.13 minutes, 386/388 (M+H)⁺

### EXAMPLE 4

### 3-Bromo-7-(2,6-difluorophenyl)-2-{[(2R)-2-(hydroxymethyl)pyrrolidin-1-yl]carbonyl}thieno-[2,3-b]pyridin-6(7H)-one (Intermediate 4)

Intermediate 3 (1.75 g, 4.53 mmol) is dissolved in dry DMF (10 mL). 1,1′-Carbonyldiimidazole (810 mg, 5.00 mmol) is added and the mixture stirred for 15 minutes at room temperature before adding (R)-2-pyrrolidinemethanol (510 mg, 5.00 mmol). The mixture is stirred at room temperature for 24 hours. More (R)-2-pyrrolidinemethanol (174 mg, 1.60 mmol) is added and the mixture allowed to stir for a further 18 hours. After concentration in vacuo, the crude reaction mixture is treated with water (100 mL) and extracted with ethyl acetate (2×100 mL). The combined organics are washed with sat. brine (100 mL) and concentrated in vacuo. The crude product is purified by chromatography (silica, dichloromethane increasing gradient with 5% steps of ethyl acetate) to give the title product as a white solid (1.64 g, 76%). δH (DMSO-d6) 7.95 (1H, d, J 9.6 Hz), 7.86-7.74 (1H, m), 7.58-7.46 (2H, m), 6.72 (1H, d, J 9.6 Hz), 4.76 (1H, t, J 5.8 Hz), 4.10-3.90 (1H, m), 3.60-3.25 (4H, m), 2.00-1.80 (4H, m). LCMS (ES+) RT 2.91 minutes, 469/471 (M+H)⁺.

Intermediates 6, 8, 10 and 18 are prepared in a similar manner to the method described in Example 4. The reagents used and the results obtained are tabulated below (Table 1).

TABLE 1

| Int. | | Starting | | ¹H NMR |
|---|---|---|---|---|
| N^{o} | IUPAC Name | Materials | LCMS | (Solvent, δ ppm) |
| 6 | 3-Bromo-7-(2,6- | Int 3 | RT 2.91 | δH (DMSO-d6) 7.95 (1H, d, J |
| | difluorophenyl)-2-{[(2S)-2- | carbonyldiimidazole | minutes469/ | 9.6 Hz), 7.86-7.74 (1H, m), |
| | (hydroxymethyl)pyrrolidin- | (S)-2- | 471 (M + H)⁺ | 7.58-7.46 (2H, m), 6.72 (1H, d, |
| | 1-yl]carbonyl}-thieno[2,3- | pyrrolidine | | J 9.6 Hz), 4.76 (1H, t, J 5.8 Hz), |
| | b]pyridin-6(7H)-one | methanol | | 4.10-3.90 (1H, m), |
| | | | | 3.60-3.25 (4H, m), 2.00-1.80 (4H, m). |
| 8 | 3-Bromo-7-(2,6- | Int 3 | RT 2.87 | δH (DMSO-d6) 7.94 (1H, d, J |
| | difluorophenyl)-2-{[(3R)-3- | carbonyldiimidazole | minutes | 9.7 Hz), 7.85-7.75 (1H, m), |
| | hydroxypyrrolidin-1- | (R)-3- | 455/ | 7.60-7.42 (2H, m), 6.72 (1H, d, |
| | yl]carbonyl}thieno[2,3- | pyrrolidinol | 457 (M + H)⁺ | J 9.7 Hz), 5.05-4.95 (1H, m), |
| | b]pyridin-6(7H)-one | | | 4.40-4.18 (1H, m), |
| | | | | 3.80-3.40 (3H, m). 3.35-3.20 (1H, m), |
| | | | | 2.02-1.82 (2H, m). |
| 10 | 3-Bromo-7-(2,6- | Int 3 | RT 2.87 | δH (DMSO-d6) 7.94 (1H, d, J |
| | difluorophenyl)-2-{[(3S)-3- | carbonyldiimidazole | minutes | 9.7 Hz), 7.85-7.75 (1H, m), |
| | hydroxypyrrolidin-1- | (S)-3- | 455/ | 7.60-7.42 (2H, m), 6.72 (1H, d, |
| | yl]carbonyl}-thieno[2,3- | pyrrolidinol | 457 (M + H)⁺ | J 9.7 Hz), 5.05-4.95 (1H, m), |
| | b]pyridin-6(7H)-one | | | 4.40-4.18 (1H, m), |
| | | | | 3.80-3.40 (3H, m). 3.35-3.20 (1H, m), |
| | | | | 2.02-1.82 (2H, m). |
| 18 | 3-Bromo-7-(2,6- | Int 3 | RT 3.58 | δH (DMSO-d6) 7.94 (1H, d, J |
| | difluorophenyl)-2-{[4-(tert- | carbonyldiimidazole | minutes, | 9.7 Hz), 7.86-7.78 (1H, m), |
| | butoxycarbonyl)piperazin- | 1-(t- | 554/ | 7.55-7.50 (2H, m), 6.74 (1H, d, |
| | 1-yl]carbonyl}-thieno[2,3- | butoxycarbonyl)piperazine | 556 (M + H)⁺ | J 9.7 Hz), 3.49-3.28 (8H, m), |
| | b]pyridin-6(7H)-one | | | 1.40 (9H, s). |

“Int.” means intermediate.
“N” means number.

### EXAMPLE 5

### 3-Bromo-7-(2,6-difluorophenyl)-2-({(2R)-2-[(tetrahydro-2H-pyran-2-yloxy) methyl]pyrrolidin-1-yl}carbonyl)thieno[2,3-b]pyridin-6(7H)-one (Intermediate 5)

Intermediate 4 (1.66 g, 3.44 mmol) is dissolved in dry dichloromethane (25 mL). 3,4-dihydro-2H-pyran (1.48 g, 17.0 mmol) and p-toluenesulfonic acid monohydrate (14 mg) are added. The mixture is stirred at room temperature for 18 hours. The mixture is treated with dichloromethane (150 mL) and washed with sat. sodium bicarbonate solution (200 mL). The organic phase is dried (Na₂SO₄) and concentrated in vacuo. The crude material is purified by chromatography (silica, DCM 85%, ethyl acetate 15% increasing gradient to 50% ethyl acetate) to give the title compound as a pale yellow solid (1.89 g, 96%). δH (DMSO-d6) 7.94 (1H, d, J 9.7 Hz), 7.84-7.77 (1H, m), 7.56-7.50 (2H, m), 6.73 (1H, d, J 9.7 Hz), 4.53-4.44 (1H, m), 4.20-4.14 (1H, m), 3.85-3.31 (6H, m), 2.05-1.30 (10H, m). LCMS (ES+) RT 3.64 minutes, 575/577 (M+Na)⁺. Intermediates 7, 9 and 11 are prepared in a similar manner to the method described in Example 5. The reagents used and the results obtained are tabulated below (Table 2).

TABLE 2

| Int. | | | | ¹H NMR |
|---|---|---|---|---|
| N^{o} | IUPAC Name | Starting Materials | LCMS | (Solvent, δ ppm) |
| 7 | 3-Bromo-7-(2,6- | Intermediate 6 | RT 3.64 | δH (DMSO-d6) 7.94 (1H, d, J |
| | difluorophenyl)- | 3,4-dihydro-2H- | minutes, | 9.7 Hz), 7.84-7.77 (1H, m), |
| | 2-({(2S)-2- | pyran | 575/ | 7.56-7.50 (2H, m), 6.73 (1H, d, J |
| | [(tetrahydro-2H- | p-toluenesulfonic | 577 (M + Na)⁺ | 9.7 Hz), 4.53-4.44 (1H, m), |
| | pyran-2- | acid | | 4.20-4.14 (1H, m), 3.85-3.31 (6H, m), |
| | yloxy)methyl]pyrrolidin- | monohydrate | | 2.05-1.30 (10H, m). |
| | 1- | | | |
| | yl}carbonyl)thieno[2, | | | |
| | 3-b]pyridin- | | | |
| | 6(7H)-one | | | |
| 9 | 3-Bromo-7-(2,6- | Intermediate 8 | RT 3.42 | δH (DMSO-d6) 7.95 (1H, d, J |
| | difluorophenyl)- | 3,4-dihydro-2H- | minutes, | 9.7 Hz), 7.85-7.75 (1H, m), |
| | 2-{[(3R)-3- | pyran | 563/ | 7.54-7.49 (2H, m), 6.74 (1H, d, J |
| | (tetrahydro-2H- | p-toluenesulfonic | 565 (M + Na)⁺ | 9.7 Hz), 4.68-4.30 (2H, m), |
| | pyran-2- | acid | | 3.77-3.30 (6H, m), 2.06-1.94 (2H, m), |
| | yloxy)pyrrolidin- | monohydrate | | 1.70-1.43 (6H, m). |
| | 1- | | | |
| | yl]carbonyl}thieno[2, | | | |
| | 3-b]pyridin- | | | |
| | 6(7H)-one | | | |
| 11 | 3-Bromo-7-(2,6- | Intermediate 10 | RT 3.42 | δH (DMSO-d6) 7.95 (1H, d, J |
| | difluorophenyl)- | 3,4-dihydro-2H- | minutes, | 9.7 Hz), 7.85-7.75 (1H, m), |
| | 2-{[(3S)-3- | pyran | 563/ | 7.54-7.49 (2H, m), 6.74 (1H, d, J |
| | (tetrahydro-2H- | (p-toluenesulfonic | 565 (M + Na)⁺ | 9.7 Hz), 4.68-4.30 (2H, m), |
| | pyran-2- | acid | | 3.77-3.30 (6H, m), 2.06-1.94 (2H, m), |
| | yloxy)pyrrolidin- | monohydrate | | 1.70-1.43 (6H, m). |
| | 1- | | | |
| | yl]carbonyl}thieno[2, | | | |
| | 3-b]pyridin- | | | |
| | 6(7H)-one | | | |

### EXAMPLE 6

### 3-Amino-7-(2,6-difluoro-phenyl)-2-{[(3R)-3-(tetrahydro-2H-pyran-2-yloxy)pyrrolidin-1-yl]carbonyl}thieno[2,3-b]pyridin-6(7H)-one (Intermediate 12)

A mixture of Sodium 3-Cyano-1-(2,6-difluorophenyl)-6-oxo-1,6-dihydropyridine-2-thiolate (RN 851749-68-3) (8.5 g, 28 mmol), 2-Chloro-1-[(R)-3-(tetrahydro-pyran-2-yloxy)-pyrrolidin-1-yl]ethanone (RN 817177-47-2, 7 g, 28 mmol) and DIPEA (5.9 mL, 33.6 mmol) in acetonitrile (85 mL) is heated under reflux for 3 h. The mixture is cooled. Water (85 mL) is added over 5 min, followed by ethyl acetate (50 mL). The phases are separated and the aqueous extracted with a further 2×50 mL of ethyl acetate. The combined organic phases are dried over MgSO₄ and concentrated. The crude product is purified by chromatography on silica gel, eluting with ethyl acetate/DCM mixtures. The title compound is obtained in 64% yield (8.57 g). δH (DMSO-d6) 8.26 (1H, d, J 9.7 Hz), 7.70-7.82 (1H, m), 7.45-7.54 (2H, m), 7.39 (1H, b), 6.60 (d, J 9.7 Hz), 4.65 (1H, m), 4.29 (1H, m), 3.33-3.78 (6H, m), 1.82-2.02 (2H, m), 1.30-1.74 (6H, m). LCMS (Conditions B) RT 3.22 minutes, 476 (M+H)⁺

### EXAMPLE 7

### 7-(2,6-Difluoro-phenyl)-3-[(5-fluoro-6-methyl-pyridin-2-yl)amino]-2-{[(3R)-3-(tetrahydro-2H-pyran-2-yloxy)pyrrolidin-1-yl]carbonyl}thieno[2,3-b]pyridin-6(7H)-one (Intermediate 13)

DME (50 mL) is degassed by bubbling nitrogen through for 0.5 h. This is added to a mixture of Intermediate 12 (3.5 g, 7.4 mmol), 2-bromo-5-fluoro-6-picoline (1.5 g, 7.7 mmol), Pd₂(dba)₃ (337 mg, 0.37 mmol), Xantphos (426 mg, 0.74 mmol) and K₃PO₄ (2.34 g, 11.0 mmol). The mixture is degassed by evacuating under reduced pressure then refilling with nitrogen, three times, then heated to reflux for 4 h. After cooling, water (25 mL) is added, and the mixture is filtered through Whatman GF/F microfibre filter paper. DCM (25 mL) is used to rinse the GF/F and then to extract the aqueous phase. The combined organic phases are washed with 25 mL of brine and then concentrated in vacuo to a thick oil. This is chromatographed on silica gel, eluting with 30% ethyl acetate in DCM. The title compound is obtained in 95% yield (4.1 g), δH (DMSO-d6) 9.51 (1H, s), 7.98 (1H, d, J 9.5 Hz), 7.72-7.86 (1H, m), 7.42-7.57 (3H, m), 6.74 (1H, dd, J 8.9, 2.8 Hz), 6.60 (1H, d, J 9.5 Hz), 4.41-4.60 (1H, m), 4.10-4.20 (1H, m), 3.12-3.74 (6H, m), 2.23 (3H, d, JH-F, 2.8 Hz), 1.20-1.82 (8H, m). LCMS (Conditions B) RT 3.75 minutes, 584 (M+H)⁺.

### EXAMPLE 8

### 3-Amino-7-(2,6-difluorophenyl)-2-{[(2R)-2-(hydroxymethyl)pyrrolidin-1-yl]carbonyl}thieno[2,3-b]pyridin-6(7H)-one (Intermediate 14)

To a 100 mL jacketed vessel is charged (R)-2-pyrrolidinemethanol (1.0 g, 9.89 mmol), N,N-diisopropylethylamine (1.9 mL, 10.88 mmol) and MeCN (10 mL). The mixture is stirred and cooled to −10° C. A solution of chloroacetyl chloride (0.79 mL, 9.89 mmol) in MeCN (2 mL) is added over 45 min using a syringe pump. The reaction is held for a further 30 min at −10° C. before warming to room temperature. Sodium 3-Cyano-1-(2,6-difluorophenyl)-6-oxo-1,6-dihydropyridine-2-thiolate (2.84 g, 9.89 mmol) and further N,N-diisopropylethylamine (1.9 mL, 10.88 mmol) are added and the reaction mixture is heated to reflux for 2 h. The mixture is cooled to room temperature and quenched by addition of water (20 mL). The mixture is extracted with DCM (3×20 mL), dried over MgSO₄, and concentrated to dryness. The crude foam obtained is crystallised from DCM (20 mL) to give a yellow solid (1.85 g), δH (DMSO-d6) 8.25 (1H, d, J 9.6 Hz), 7.75 (1H, m), 7.49 (2H, t, J 8.9 Hz), 7.33 (1H, m), 6.59 (1H, d, J 9.6 Hz), 4.71 (1H, t, J 5.7 Hz), 4.14 (1H, m), 3.48 (3H, m), 3.29 (1H, m), 1.80 (4H, m). LCMS (Conditions B) (ES+) 406 (M+H)⁺.

### EXAMPLE 9

### 3-Amino-7-(2,6-difluorophenyl)-2-({(2R)-2-[tetrahydro-2H-pyran-2-yloxy) methyl]pyrrolidin-1-yl}carbonyl)thieno[2,3-b]pyridin-6(7H)-one (Intermediate 15)

To a suspension of intermediate 14 (2.25 g, 5.56 mmol) in DCM (45 mL) at 0° C. is charged 3,4-dihydro-2H-pyran (1.01 mL, 11.11 mmol) and p-toluenesulfonic acid monohydrate (20 mg, 0.11 mmol). The mixture is stirred at 0° C. for 24 h before quenching by addition of saturated aqueous sodium bicarbonate solution (20 mL) and warming to room temperature. The DCM layer is separated, dried over MgSO₄, and concentrated to dryness. The crude product obtained is purified by column chromatography (0-50% EtOAc/heptane) to give a complex mixture of diastereomers as a yellow solid (2.44 g), LCMS (Conditions B) (ES+) 512 (M+Na)⁺.

### EXAMPLE 10

### 7-(2,6-Difluorophenyl)-3-[(5-fluoro-6-methylpyridin-2-yl)amino]-2-({(2R)-2-[(tetrahydro-2H-pyran-2-yloxy)methyl]pyrrolidin-1-yl}carbonyl)thieno[2,3-b]pyridin-6(7H)-one (Intermediate 16)

To a 100 mL pre dried three-necked round bottomed flask is charged Intermediate 15 (500 mg, 1.02 mmol), Pd₂(dba)₃ (47 mg, 0.051 mmol), Xanthphos (56 mg, 0.10 mmol), K₃PO₄ (325 mg, 1.53 mmol), 2-bromo-5-fluoro-6-picoline (232 mg, 1.22 mmol), and DME (20 mL). The mixture is degassed by performing a three evacuate & re-fill cycles with nitrogen and then heated to reflux for 6 h. The mixture is cooled to room temperature, water (20 mL) is added and the mixture is extracted with DCM (3×20 mL). The organic layer is dried over MgSO₄ and concentrated to dryness. The crude product obtained is used directly in the preparation of Compound 6 without purification.

### EXAMPLE 11

### 7-(2,6-Difluorophenyl)-2-{[4-(tert-butoxycarbonyl)piperazin-1-yl]carbonyl}-3-(6-methylpyridin-2-ylamino)thieno[2,3-b]pyridin-6(7H)-one (Intermediate 17)

Intermediate 18 (600 mg, 1.08 mmol) is dissolved in dry toluene (5 mL) and the following reagents added, 2-amino-6-picoline (141 mg, 1.30 mmol), caesium carbonate (492 mg, 1.51 mmol), tris-(dibenzylideneacetone)dipalladium(0) (49 mg, 0.054 mmol) and rac-BINAP (67 mg, 0.018 mmol). The reaction mixture is heated to 100° C. in a sealed tube for 18 hours. The mixture is cooled and partitioned between water (75 mL) and dichloromethane (100 mL). The aqueous is further extracted with dichloromethane (2×100 mL) and the combined organics dried (MgSO₄) and concentrated in vacuo. Purification by chromatography (silica, 25-35% EtOAc in dichloromethane) gives the title compound as a pale brown solid (300 mg, 48%). δH (DMSO-d6) 9.35 (1H, brs), 8.00 (1H, d, J 9.7 Hz), 7.84-7.74 (1H, m), 7.54-7.47 (3H, m), 6.68-6.64 (2H, m), 6.62 (1H, d, J 9.7 Hz), 3.19-3.17 (4H, m), 3.09-3.06 (4H, m), 2.23 (3H, s), 1.35 (9H, s). LCMS (ES⁺) RT 3.18 minutes, 582 (M+H)⁺

### EXAMPLE 12

### 7-(2,6-difluorophenyl)-3-{[6-(hydroxmethyl)pyridin-2-yl]amino}-2-{[(3R)-3-(tetrahydro-2H-pyran-2-yloxy)pyrrolidin-1-yl]carbonyl}thieno[2,3-b]pyridin-6(7H)-one (Intermediate 19)

A 20 mL microwave vial is charged with a solution of Intermediate 9 (1.8 g, 3.34 mmol) in anhydrous, degassed DME (18 mL, 10 volumes) to which is added 2-amino-6-(hydroxymethyl)pyridine (RN 79651-64-2, 497 mg, 4.0 mmol), tris(dibenzylideneacetone)dipalladium(0) (5 mol %), rac-BINAP (10 mol %) and cesium carbonate (1.52 g, 4.68 mmol). The whole mixture is degassed for an extra 10 min by sparging with nitrogen before being microwaved for 90 minutes (120° C., 300 W). The mixture is filtered and fresh reagents: tris(dibenzylideneacetone)dipalladium (0) (5 mol %), rac-BINAP (10 mol %) and cesium carbonate (1.52 g, 4.68 mmol) added. The whole mixture is degassed for 10 min before being microwaved for a further 60 min (120° C., 300 W). Once the reaction is complete the brown mixture is filtered through celite and rinsed with DME (2×1.8 mL, 2 vols). This solution is concentrated to dryness to give the intermediate 7-(2,6-difluorophenyl)-3-{[6-(hydroxymethyl)pyridin-2-yl]amino}-2-{[(3R)-3-(tetrahydro-2H-pyran-2-yloxy)pyrrolidin-1-yl]carbonyl}thieno[2,3-b]pyridin-6(7H)-one as a crude oil, which can be taken on to the next step without purification. If desired this intermediate can be purified by preparative HPLC resulting in a yellow solid.

δH (D₆-DMSO, 300 MHz) 9.57 (1H, s, —NH), 7.98 (1H, dd, —CH—CH—CO), 7.79 (1H, m, Ar—H), 7.65-7.45 (3H, m, Ar—H), 6.89 (1H, d, Pyridine-CH), 6.75 (1H, d, Pyridine-CH), 6.61 (1H, d, —CH—CO), 4.50 (1H, s, —O—CH—O), 4.32 (2H, s, —CH₂—O), 4.12 (2H, m, —CH—O/N), 3.30 (5H, m, —CH-0/N), 3.16 (1H, s, —OH/—NH), 1.85 (8H, s, —CH₂). LCMS (ES+) RT 3.13 mins (method B), 583.0 (M+H)⁺

### EXAMPLE 13

### 7-(2,6-Difluorophenyl)-2-{[(2R)-2-(hydroxymethyl)pyrrolidin-1-yl]carbonyl}-3-[6-methylpyridin-2-yl)amino]thieno-[2,3-b]pyridin-6(7H)-one (Compound 1)

Intermediate 5 (1.00 g, 1.80 mmol) is dissolved in dry toluene (10 mL) and the following reagents added, 2-amino-6-picoline (240 mg, 2.17 mmol), caesium carbonate (880 mg, 2.70 mmol), Pd₂(dba)₃ (84 mg, 0.09 mmol) and rac-BINAP (112 mg, 0.18 mmol). The reaction mixture is heated to 90° C. in a sealed tube for 18 hours. The mixture is cooled and partitioned between water (75 mL) and dichloromethane (100 mL). The aqueous is further extracted with dichloromethane (2×100 mL) and the combined organics dried (MgSO₄) and concentrated in vacuo. Chromatography (silica, dichloromethane 85%, ethyl acetate 15%) gives the intermediate THP-protected compound as a white solid (730 mg). This material is dissolved in methanol (10 mL) and 2M HCl (2 mL) is added. After stirring at room temperature for 4 hours the mixture is treated with water (80 mL) and extracted with ethyl acetate (2×100 mL). The organics are dried (Na₂SO₄) and concentrated in vacuo. The crude product is purified by chromatography (silica, dichloromethane 40%, ethyl acetate 60%) to give the title compound as a pale yellow solid (391 mg, 58%). δH (DMSO-d6) 9.42 (1H, brs), 8.05 (1H, d, J 9.6 Hz), 7.90-7.80 (1H, m), 7.60-7.50 (3H, m), 6.75-6.61 (3H, m), 4.73-4.63 (1H, m), 4.05-3.95 (1H, m), 3.45-3.30 (3H, m), 3.07-2.95 (1H, m), 2.32 (3H, s), 1.70-1.42 (4H, m). LCMS (ES+) RT 2.43 minutes, 497 (M+H)⁺

Compounds 2, 3 and 4 are prepared in a similar manner to the method described in Example 12. The reagents used and the results obtained are tabulated below (Table 3).

TABLE 3

| | | | | ¹H NMR |
|---|---|---|---|---|
| Cpd | | Starting | | (Solvent, |
| N^{o} | IUPAC Name | Materials | LCMS | δ ppm) |
| 2 | 7-(2,6- | Int 7 | RT 2.43 | δH (DMSO-d6) 9.42 (1H, |
| | Difluorophenyl)-2- | 2-amino-6- | minutes, | brs), 8.05 (1H, d, J 9.6 Hz), |
| | {[(2S)-2- | picoline | 497 (M + H)+ | 7.90-7.80 (1H, m), |
| | (hydroxymethyl)pyrrolidin- | caesium | | 7.60-7.50 (3H, m), |
| | 1-yl]carbonyl}- | carbonate | | 6.75-6.61 (3H, m), 4.73-4.63 (1H, m), |
| | 3-[(6-methylpyridin- | tris- | | 4.05-3.95 (1H, m), |
| | 2- | (dibenzylidene | | 3.45-3.30 (3H, m), |
| | yl)amino]thieno[2,3- | acetone) | | 3.07-2.95 (1H, m), 2.32 (3H, s), |
| | b]pyridin-6(7H)-one | palladium (0) | | 1.70-1.42 (4H, m). |
| | | rac-BINAP | | |
| 3 | 7-(2,6- | Int 9 | RT 2.25 | δH (DMSO-d6) 9.46 (1H, |
| | Difluorophenyl)-2- | 2-amino-6- | minutes, | brs), 7.88 (1H, d, J 9.7 Hz), |
| | {[(3R)-3- | picoline | 483 (M + H)+ | 7.84-7.74 (1H, m), |
| | hydroxypyrrolidin-1- | caesium | | 7.54-7.46 (3H, m), |
| | yl]carbonyl}-3-[(6- | carbonate | | 6.68-6.64 (2H, m), 6.58 (1H, d, J |
| | methylpyridin-2- | (tris- | | 9.7 Hz), 4.83 (1H, d, J |
| | yl)amino]thieno[2,3- | (dibenzylidene | | 2.6 Hz), 3.46-3.30 (3H, m), |
| | b]pyridin-6(7H)-one | acetone) | | 3.17-3.12 (1H, m), |
| | | palladium (0) | | 2.26 (3H, s), 1.68-1.62 (2H, m). |
| | | rac-BINAP | | |
| 4 | 7-(2,6- | Int 11 | RT 2.25 | δH (DMSO-d6) 9.46 (1H, |
| | Difluorophenyl)-2- | 2-amino-6- | minutes, | brs), 7.88 (1H, d, J 9.7 Hz), |
| | {[(3S)-3- | picoline | 483 (M + H)+ | 7.84-7.74 (1H, m), |
| | hydroxypyrrolidin-1- | caesium | | 7.54-7.46 (3H, m), |
| | yl]carbonyl}-3-[(6- | carbonate tris- | | 6.68-6.64 (2H, m), 6.58 (1H, d, J |
| | methylpyridin-2- | (dibenzylidene | | 9.7 Hz), 4.83 (1H, d, J |
| | yl)amino]thieno[2,3- | acetone) | | 2.6 Hz), 3.46-3.30 (3H, m), |
| | b]pyridin-6(7H)-one | palladium (0) | | 3.17-3.12 (1H, m), |
| | | rac-BINAP | | 2.26 (3H, s), 1.68-1.62 (2H, m). |

“Cpd” means compound.
“Int” means Intermediate

### EXAMPLE 14

### 7-(2,6-Difluoro-phenyl)-3-[(5-fluoro-6-methylpyridin-2-yl)amino]-2-{[(3R)-3-hydroxypyrrolidin-1-yl]carbonyl}thieno[2,3-b]pyridin-6(7H)-one (Compound 5)

A mixture of Intermediate 13 (4 g, 6.8 mmol), ethanol (60 mL), c.HCl (1.1 mL, 13.6 mmol), and water (6.9 mL) is stirred at r.t. overnight. Sodium hydroxide solution (3M) is then added to pH 6, followed by DCM (100 mL) and water (20 mL). The phases are separated, the organic phase is washed with brine (25 mL), dried over MgSO₄ and concentrated in vacuo. The crude product is purified by chromatography on silica gel, elution with ethanol/DCM mixtures. The fractions are concentrated to a thick oil, then 50 mL acetonitrile is added and the mixture re-concentrated to encourage crystallisation. This is repeated twice. The title compound is obtained as a pale yellow solid (2.6 g, 76%). δH (DMSO-d6) 9.51 (1H, s), 7.92 (1H, d, J 9.5 Hz), 7.73-7.84 (1H, m), 7.43-7.57 (3H, m), 6.73 (1H, dd, J 8.9, 2.5 Hz), 6.59 (1H, d, J 9.5 Hz), 4.86 (1H, d, J 3.2 Hz), 4.05-4.16 (1H, m), 3.21-3.46 (3H, m), 3.05-3.16 (1H, m), 2.23 (3H, d, JH-F 2.5 Hz), 1.58-1.70 (2H, m).

LCMS (Conditions B) RT 3.75 minutes, 501 (M+H)⁺, Differential Scanning Calorimetry (DSC) Tonset, 239.3° C., Tmax, 241.1° C., 165 J/g.

### EXAMPLE 15

### 7-(2,6-Difluorophenyl)-3-[(5-fluoro-6-methylpyridin-2-yl)amino]-2-{[(2R)-2-(hydroxymethyl)pyrrolidin-1-yl]carbonyl}thieno[2,3-b]pyridin-6(7H)-one (Compound 6)

To a solution of crude intermediate 16 (1.4 g, 2.34 mmol) in EtOH (28 mL) at room temperature is added HCl (1M, 9.36 mL). The reaction mixture is stirred at room temperature for 16 h. Upon completion of the reaction the mixture is neutralised by dropwise addition of 2M NaOH(aq) to pH 8.5. EtOH is removed in vacuo and the aqueous mixture is extracted with DCM (3×20 mL). The combined organic layers are dried over MgSO₄, and concentrated to dryness. The crude oil obtained is purified by column chromatography (0-100% EtOAc/DCM) to give the title compound as a white solid (101 mg). δH (DMSO-d6) 9.43 (1H, s), 7.94 (1H, m), 7.8 (1H, m), 7.51 (3H, m), 6.70 (1H, m), 6.65 (2H, m), 4.68 (1H, m), 3.92 (1H, m), 3.25 (2H, m), 2.95 (1H, m), 2.25 (3H, s), 1.6 (4H, m). LCMS (Conditions B) (ES+) 514 (M+H)⁺.

### EXAMPLE 16

### 7-(2,6-Difluorophenyl)-3-(6-methylpyridin-2-ylamino)-2-{[1-piperazinyl]carbonyl}thieno[2,3-b]pyridin-6(7H)-one (Compound 7)

Intermediate 18 (270 mg, 0.50 mmol) is dissolved in 4.0M HCl in dioxane (7 mL) and ethanol (6 mL). The mixture is stirred at room temperature for 4 hours. The solvent is removed in vacuo and the resultant solids partitioned between dichloromethane (30 mL) and 0.1M NaOH solution (30 mL). The organic is dried (Na₂SO₄) and concentrated in vacuo. Chromatography (silica, 2% to 12% methanol gradient in dichloromethane) gives the title compound as a pale yellow solid (125 mg, 56%). δH (DMSO-d6) 9.33 (1H, brs), 7.99 (1H, d, J 9.7 Hz), 7.82-7.74 (1H, m), 7.54-7.46 (3H, m), 6.67-6.64 (2H, m), 6.60 (1H, d, J 9.7 Hz), 3.17-3.14 (4H, m), 2.46-2.42 (4H, m), 2.23 (3H, s). LCMS (ES⁺) RT 1.71 minutes, 482 (M+H)⁺.

### EXAMPLE 17

### 7-(2,6-Difluorophenyl)-3-{[6-(hydroxymethyl)pyridin-2-yl]amino}-2-{[(3R)-3-hydroxypyrrolidin-1-yl]carbonyl}thieno[2,3-b]pyridin-6(7H)-one (Compound 8)

To a suspension of the crude intermediate 19 (3.89 g theoretical maximum from combining two 1.8 g microwave batches, 6.7 mmol) in DCM (46.7 ml, 12 vols) in a three necked flask fitted with an overhead mechanical stirrer is added an aqueous solution of hydrochloric acid (0.5M, 13 vols, 0.02M). The resultant biphasic mixture is stirred for 4 h at room temperature. The phases are separated and the aqueous acidic phase (pH=0) neutralised to pH=12 using 5M NaOH (6.5 mL) with formation of a precipitate in the solution. The suspension is stirred at 0° C. for 1 hour before being filtered and washed with cold water (1 vol). The product is sucked dry and dried in the vacuum oven before re-crystallisation from EtOAc (10 vols) at 50° C. The product is filtered and dried at 40° C. to give the title compound as a beige solid (0.735 g, 22% over two steps). δH (CDCl₃, 400 MHz) 9.55 (1H, s, —NH), 7.75 (1H, d, J=10 Hz, —CH—CH—CO), 7.55 (2H, m, Ar—H), 7.20 (1H, m, ArH), 7.50 (2H, m, Ar—H), 6.80 (1H, d, J=7 Hz, Pyridine-CH), 6.75 (1H, d, J=8 Hz, Pyridine-CH), 6.55 (1H, d, J=9.5 Hz, —CH—CO), 4.60 (2H, s, —CH₂—O), 4.50 (1H, wide peak, —CH—O), 3.75 (2H, m, —CH₂—N), 3.65 (2H, m, —CH₂—N), 2.00 (2H, m, —CH—CH₂). LCMS (Method B) RT 3.73 mins.

The following assays and animal models are used to demonstrate the potency and selectivity of the compounds according to the invention. In each assay an IC50 value is determined for each test compound and represents the concentration of compound necessary to achieve 50% inhibition.

### EXAMPLE 18

### Preparation of Activated Human p38α for Inhibitor Assays

a). Purification of Human p38α

Human p38α, incorporating an N-terminal (His)6 tag, is expressed in baculovirus-infected High-Five™ cells (Invitrogen) according to the manufacturers instructions. The cells are harvested 72 h post-infection and lysed in phosphate buffered saline (PBS) containing 1% (w/v) β-octylglucoside and Complete, EDTA-free™ protease inhibitors (Roche Molecular Biochemicals). The lysate is centrifuged at 35000×g for 30 min at 4° C. and the supernatant applied to a NiNTA™ column (Qiagen). Bound protein is eluted by 150 mM imidazole in PBS (after a wash with 15 mM imidazole in PBS) and directly applied to a HiTrap Q™ column (AP Biotech). Bound protein is eluted using a 20 column volume, 0 to 1M NaCl gradient. Fractions containing (His)6-p38 are aliquotted and stored at −70° prior to their activation.

### b). Preparation of GST-MKK6EE-Containing Lysates

E. coli (BL21 pLysS) expressing the constitutively activated form of human MKK6 fused with an N-terminal glutathione-S-transferase tag (GST-MKK6EE) are harvested by centrifugation and frozen at −70°. Cells are lysed by resuspension in 1/10th the culture volume of PBS containing Complete, EDTA-free™ protease inhibitors followed by sonication on ice for 4×15 sec. Cell debris is removed by centrifugation at 35,000×g and the resultant supernatant stored in aliquots at −70°.

### c). Activation of (His)6-p38

0.45 mL of purified (His)6-p38 is incubated with 50 μL of the GST-MKK6EE-containing lysate for 30 min at 23° in the presence of 1 mM β-glycerophosphate, 10 mM MgCl₂ and 9 mM ATP. The extent of activation is monitored by mass spectrometric detection of the doubly-phosphorylated form of (His)6-p38, which routinely comprised greater than 90% of the final (His)6-p38 preparation. The activated (His)6-p38 is then diluted ×10 in PBS and repurified using the method described above. The concentration of purified, activated (His)6-p38 is measured by UV absorbance at 280 nm using A280, 0.1%=1.2 and the preparation stored in aliquots at −70° prior to its use in inhibitor assays.

### EXAMPLE 19

### p38 Inhibition Assays

### a). Inhibition of Phosphorylation of Biotinylated Myelin Basic Protein (MBP)

The inhibition of p38 catalysed phosphorylation of biotinylated MBP is measured using a DELFIA based format. The assay is performed in a buffer comprising, 20 mM HEPES (pH 7.4), 5 mM MgCl₂ and 3 mM DTT. For a typical IC50 determination, biotinylated MBP (2.5 μM) is incubated at room temperature in a streptavidin-coated microtitre plate together with activated gst-p38 (10 nM) and ATP (10/1) in the presence of a range of inhibitor concentrations (final concentration of DMSO is 2 percent). After fifteen minutes the reaction is terminated by the addition of EDTA (75 mM). The microtitre plate is then washed with Tris buffered saline (TBS), prior to the addition of 100 μl of anti-phospho MBP antibody (mouse) together with europium-labelled anti-mouse IgG antibody. After one hour at room temperature the plate is washed again in TBS followed by the addition of Enhancement solution (PerkinElmer Wallac). Fluorescence measurements are performed after a further fifteen minutes at room temperature. IC50 values are determined from the plot of Log₁₀, inhibitor concentration (x-axis) versus percentage inhibition of the fluorescence generated by a control sample in the absence of inhibitor (y-axis).

The compounds of the invention are tested in this assay (p38 MAP Kinase) and show IC50' s between 5 nM and 500 nM.

Compound 1 inhibits p38α MAP kinase with IC50 between 15 nM and 30 nM.

b). Purification of Human Peripheral Blood Mononuclear Cells Peripheral blood mononuclear cells (PBMC) are isolated from normal healthy volunteers. Whole blood is taken by venous puncture using heparinised vacutainers (Becton Dickinson), diluted 1 in 3 in RPMI 1640 (Gibco, UK) and centrifuged at 400 g for 35 min over a Ficoll-paque gradient (Amersham-Pharmacia Biotech, UK). Cells at the interface are removed and washed once followed by a low speed spin (250 g) to remove platelets. Cells are then resuspended in DMEM containing 10% FCS and glutamine 2 mM (Gibco, UK).

### c). Inhibitor Dilutions

Inhibitor stocks (10 mM) are kept as a frozen solution (−20° C.) in DMSO. Serial dilutions of inhibitors are prepared in DMSO as 200-times concentrated stocks. Inhibitors are diluted 1 in 200 into tissue culture media, prewarmed to 37° C. and transferred to plates containing PBMC. PBMC and inhibitors are incubated together for 30 mins prior to addition of LPS. Inhibitors used in whole blood assays are prepared according to a different regime. Using the same stock solution, serial dilutions of inhibitors are prepared in DMSO. Inhibitors (14) are then diluted 1 in 500 straight into whole blood. Inhibitor is incubated with whole blood for 30 mins prior to the addition of LPS.

### d). LPS Stimulation of PBMC

PBMC are resuspended at a density of 2×10⁵ cells/well in flat bottomed 96 well tissue culture treated plates. After the addition of inhibitor cells are stimulated with an optimal dose of LPS (E coli strain B5:055, Sigma, at a final concentration of 1 μg ml⁻¹) and incubated at 37° C. in 5% CO₂/95% air for 18 hours. TNF-α levels are measured from cell free supernatants by sandwich ELISA (R&D Systems, # DY210).

### e). LPS Stimulation of Whole Blood

Whole blood is taken by venous puncture using heparinised vacutainers (Becton Dickinson), and 500 μl of blood aliquoted into each well of a 96 deep well plate. After the addition of inhibitor, blood is stimulated with an optimal dose of LPS (E coli strain B5:055, Sigma, at a final concentration of 1 μg ml⁻¹) and incubated at 37° C. without CO₂ for 18 hours. TNF-α levels are measured from cell free supernatants by sandwich ELISA (R&D Systems, # DY210).

The compounds of the invention are tested in this assay and the LPS stimulated release of TNF from Human whole blood IC50 is between 15 nM and 5 μM. Compound 1 inhibits this assay with IC50 between 30 nM and 200 nM (n=12).

### f). Rat LPS Induced TNF Release

Male Lewis rats (180-200 g) are anaesthetized with Isofluor and injected i.v. with LPS (1 mg/kg) in a volume of 0.5 ml sterile saline. After 90 minutes blood is collected into heparin tubes for preparation of plasma samples. Plasma is stored at −80° C. prior to assay for TNFα by commercial ELISA.

The compounds of the invention are tested in this assay and the LPS stimulated TNF release in rat and the ED50 is less than 30 mg/kg dosed p.o. Compound 1 inhibits this model with ED50<10 mg/kg p.o.

### g). Rat Collagen Induced Arthritis (CIA)

Female Lewis rats (180-200 g) are anaesthetized with Isofluor and immunised i.d. at the base of the tail with 2×100 μl of emulsion containing 4 mg/ml bovine collagen II in 0.01M acetic acid and Freund's Incomplete Adjuvant at a ratio of 1:1. A polyarthritis develops with onset from about 13 days post sensitisation. The disease is mainly confined to the ankles and is quantified by plethysmometry. Results are expressed as change in paw volume over time.

The compounds of the invention are tested in this assay and the CIA ED50 for rat is less than 100 mg/kg dosed po.

1. A compound having formula I or pharmaceutically acceptable salts thereof or stereoisomeric forms thereof, and the geometrical isomers, enantiomers, diastereoisomers, and pharmaceutically acceptable salts thereof 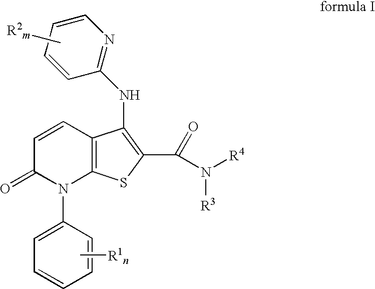
wherein
R¹ is independently C₁₋₃ alkyl, halogen or hydroxyl;
R² is independently C₁₋₃ alkyl, halogen or hydroxyl;
n is 1 to 3;
m is 1 to 3;
R³ and R⁴ form together with the nitrogen atom a 4, 5 or 6 membered non-aromatic heterocycle optionally substituted by a substituent selected from the group constituted of C₁₋₃ alkyl or hydroxyl.

2. The compound according to claim 1 wherein n is 2.

3. The compound according to claim 1 wherein R¹ is fluorine.

4. The compound according to claim 1 wherein R² is methyl, fluorine; and R¹ is fluorine; and n is 2; and R³ and R⁴ are (2R)-2-(hydroxymethyl)pyrrolidine, (2S)-2-(hydroxymethyl)pyrrolidine, (3R)-3-hydroxypyrrolidine, (3S)-3-hydroxypyrrolidine or piperazine.

5. The compound according to claim 1 that is
7-(2,6-Difluorophenyl)-2-{[(2R)-2-(hydroxymethyl)pyrrolidin-1-yl]carbonyl}-3-[(6-methylpyridin-2-yl)amino]thieno[2,3-b]pyridin-6(7H)-one;
7-(2,6-Difluorophenyl)-2-{[(2S)-2-(hydroxymethyl)pyrrolidin-1-yl]carbonyl}-3-[(6-methylpyridin-2-yl)amino]thieno[2,3-b]pyridin-6(7H)-one;
7-(2,6-Difluorophenyl)-2-{[(3R)-3-hydroxypyrrolidin-1-yl]carbonyl}-3-[(6-methylpyridin-2-yl)amino]thieno[2,3-b]pyridin-6(7H)-one;
7-(2,6-Difluorophenyl)-2-{[(3S)-3-hydroxypyrrolidin-1-yl]carbonyl}-3-[(6-methylpyridin-2-yl)amino]thieno[2,3-b]pyridin-6(7H)-one;
7-(2,6-Difluoro-phenyl)-3-[(5-fluoro-6-methylpyridin-2-yl)amino]-2-{[(3R)-3-hydroxypyrrolidin-1-yl]carbonyl}thieno[2,3-b]pyridin-6(7H)-one;
7-(2,6-Difluorophenyl)-3-[(5-fluoro-6-methylpyridin-2-yl)amino]-2-{[(2R)-2-(hydroxymethyl)pyrrolidin-1-yl]carbonyl}thieno[2,3-b]pyridin-6(7H)-one; or
7-(2,6-Difluorophenyl)-3-(6-methylpyridin-2-ylamino)-2-{[1-piperazinyl]carbonyl}thieno[2,3-b]pyridin-6(7H)-one.

6. The compound according to claim 1 that is
7-(2,6-Difluorophenyl)-2-{[(2R)-2-(hydroxymethyl)pyrrolidin-1-yl]carbonyl}-3-[(6-methylpyridin-2-yl)amino]thieno[2,3-b]pyridin-6(7H)-one;
7-(2,6-Difluorophenyl)-2-{[(3R)-3-hydroxypyrrolidin-1-yl]carbonyl}-3-[(6-methylpyridin-2-yl)amino]thieno[2,3-b]pyridin-6(7H)-one;
7-(2,6-Difluoro-phenyl)-3-[(5-fluoro-6-methylpyridin-2-yl)amino]-2-{[(3R)-3-hydroxypyrrolidin-1-yl]carbonyl}thieno[2,3-b]pyridin-6(7H)-one; or
7-(2,6-Difluorophenyl)-3-[(5-fluoro-6-methylpyridin-2-yl)amino]-2-{[(2R)-2-(hydroxymethyl)pyrrolidin-1-yl]carbonyl}thieno[2,3-b]pyridin-6(7H)-one.

7. A compound that is
Ethyl 3-amino-7-(2,6-difluorophenyl)-6-oxo-6,7-dihydrothieno[2,3-b]-pyridine-2-carboxylate;
Ethyl 3-bromo-7-(2,6-difluorophenyl)-6-oxo-6,7-dihydrothieno[2,3-b]-pyridine-2-carboxylate;
3-Bromo-7-(2,6-difluorophenyl)-6-oxo-6,7-dihydrothieno[2,3-b]pyridine-2-carboxylic acid;
3-Bromo-7-(2,6-difluorophenyl)-2-{[(2R)-2-(hydroxymethyl)pyrrolidin-1-yl]carbonyl}thieno[2,3-b]pyridin-6(7H)-one;
3-Bromo-7-(2,6-difluorophenyl)-2-({(2R)-2-[(tetrahydro-2H-pyran-2-yloxy)methyl]pyrrolidin-1-yl}carbonyl)thieno[2,3-b]pyridin-6(7H)-one;
3-Bromo-7-(2,6-difluorophenyl)-2-{[(2S)-2-(hydroxymethyl)pyrrolidin-1-yl]carbonyl}-thieno[2,3-b]pyridin-6(7H)-one;
3-Bromo-7-(2,6-difluorophenyl)-2-({(2 S)-2-[(tetrahydro-2H-pyran-2-yloxy)methyl]pyrrolidin-1-yl}carbonyl)thieno[2,3-b]pyridin-6(7H)-one;
3-Bromo-7-(2,6-difluorophenyl)-2-{[(3R)-3-hydroxypyrrolidin-1-yl]carbonyl}thieno[2,3-b]pyridin-6(7H)-one;
3-Bromo-7-(2,6-difluorophenyl)-2-{[(3R)-3-(tetrahydro-2H-pyran-2-yloxy)pyrrolidin-1-yl]carbonyl}thieno[2,3-b]pyridin-6(7H)-one;
3-Bromo-7-(2,6-difluorophenyl)-2-{[(3S)-3-hydroxypyrrolidin-1-yl]carbonyl}-thieno[2,3-b]pyridin-6(7H)-one;
3-Bromo-7-(2,6-difluorophenyl)-2-{[(3S)-3-(tetrahydro-2H-pyran-2-yloxy)pyrrolidin-1-yl]carbonyl}thieno[2,3-b]pyridin-6(7H)-one;
3-Amino-7-(2,6-difluoro-phenyl)-2-{[(3R)-3-(tetrahydro-2H-pyran-2-yloxy)pyrrolidin-1-yl]carbonyl}thieno[2,3-b]pyridin-6(7H)-one;
7-(2,6-Difluoro-phenyl)-3-[(5-fluoro-6-methyl-pyridin-2-yl)amino]-2-{[(3R)-3-(tetrahydro-2H-pyran-2-yloxy)pyrrolidin-1-yl]carbonyl}thieno[2,3-b]pyridin-6(7H)-one;
3-Amino-7-(2,6-difluorophenyl)-2-{[(2R)-2-(hydroxymethyl)pyrrolidin-1-yl]carbonyl}thieno[2,3-b]pyridin-6(7H)-one;
3-Amino-7-(2,6-difluorophenyl)-2-({(2R)-2-[(tetrahydro-2H-pyran-2-yloxy)methyl]pyrrolidin-1-yl}carbonyl)thieno[2,3-b]pyridin-6(7H)-one;
7-(2,6-Difluorophenyl)-3-[(5-fluoro-6-methylpyridin-2-yl)amino]-2-({(2R)-2-[(tetrahydro-2H-pyran-2-yloxy)methyl]pyrrolidin-1-yl}carbonyl)thieno[2,3-b]pyridin-6(7H)-one;
7-(2,6-Difluorophenyl)-2-{[4-(tert-butoxycarbonyl)piperazin-1-yl]carbonyl}-3-(6-methylpyridin-2-ylamino)thieno[2,3-b]pyridin-6(7H)-one;
3-Bromo-7-(2,6-difluorophenyl)-2-{[4-(tert-butoxycarbonyl)piperazin-1-yl]carbonyl}-thieno[2,3-b]pyridin-6(7H)-one; or
7-(2,6-difluorophenyl)-3-{[6-(hydroxymethyl)pyridin-2-yl]amino}-2-{[(3R)-3-(tetrahydro-2H-pyran-2-yloxy)pyrrolidin-1-yl]carbonyl}thieno[2,3-b]pyridin-6(7H)-one.

8. (canceled)

9. A pharmaceutical composition comprising a therapeutically effective amount of a compound according to claim 1 and a pharmaceutically acceptable adjuvant, diluent or carrier.

10. (canceled)

11. (canceled)

12. (canceled)

13. (canceled)

14. (canceled)

15. A method of treating rheumatoid arthritis comprising administering an effective amount of a compound according to claim 1.

16. The method according to claim 15 wherein the compound is
7-(2,6-Difluorophenyl)-2-{[(2R)-2-(hydroxymethyl)pyrrolidin-1-yl]carbonyl}-3-[(6-methylpyridin-2-yl)amino]thieno[2,3-b]pyridin-6(7H)-one;
7-(2,6-Difluorophenyl)-2-{[(3R)-3-hydroxypyrrolidin-1-yl]carbonyl}-3-[(6-methylpyridin-2-yl)amino]thieno[2,3-b]pyridin-6(7H)-one;
7-(2,6-Difluoro-phenyl)-3-[(5-fluoro-6-methylpyridin-2-yl)amino]-2-{[(3R)-3-hydroxypyrrolidin-1-yl]carbonyl}thieno[2,3-b]pyridin-6(7H)-one; or
7-(2,6-Difluorophenyl)-3-[(5-fluoro-6-methylpyridin-2-yl)amino]-2-{[(2R)-2-(hydroxymethyl)pyrrolidin-1-yl]carbonyl}thieno[2,3-b]pyridin-6(7H)-one.
